SEQUENCE LISTING (8 sequences)

SEQ ID NO: 1 - DNA, 33 bp, synthetic construct
  source 1..33: mol_type = other DNA
tggacatctcgggtgccaaggaactcacgtcac

SEQ ID NO: 2 - DNA, 33 bp, synthetic construct
  source 1..33: mol_type = other DNA
gtgacgtgagttccttggcacccgagatgtcca

SEQ ID NO: 3 - RNA, 22 nt, synthetic construct
  source 1..22: mol_type = other RNA
cgtgtatttgacaagctgagtt

SEQ ID NO: 4 - RNA, 22 nt, synthetic construct
  source 1..22: mol_type = other RNA
tgtcagtttgtcaaatacccca

SEQ ID NO: 5 - RNA, 22 nt, synthetic construct
  source 1..22: mol_type = other RNA
tattgcacttgtcccggcctgt

SEQ ID NO: 6 - RNA, 22 nt, synthetic construct
  source 1..22: mol_type = other RNA
gggtggggatttgttgcattac

SEQ ID NO: 7 - RNA, 22 nt, synthetic construct
  source 1..22: mol_type = other RNA
gctcttttcacattgtgctact

SEQ ID NO: 8 - RNA, 22 nt, synthetic construct
  source 1..22: mol_type = other RNA
cagtgcaatgttaaaagggcat

(19) United States
(12) Patent Application Publication
(10) Pub. No.: US 2026/0152791 A1
(43) Pub. Date: Jun. 4, 2026

(54) SEQUENCING METHOD

(71) Applicant: GENEMIND BIOSCIENCES COMPANY LIMITED, Shenzhen (CN)

(72) Inventors: Jicai FAN, Shenzhen (CN); Zhao LI, Shenzhen (CN); Pengwei XU, Shenzhen (CN); Huan JIN, Shenzhen (CN); Xuejiao YOU, Shenzhen (CN); Lei LIU, Shenzhen (CN); Lei SUN, Shenzhen (CN)

(73) Assignee: GENEMIND BIOSCIENCES COMPANY LIMITED, Shenzhen (CN)

(21) Appl. No.: 19/396,406

(22) Filed: Nov. 21, 2025

(30) Foreign Application Priority Data
Dec. 3, 2024 (CN) 202411764236.2

(51) Int. Cl. C12Q 1/6874; C12N 15/10; C12Q 1/6806; C12Q 1/6855

(52) U.S. Cl. CPC C12Q 1/6874; C12N 15/1096; C12Q 1/6806; C12Q 1/6855

(57) ABSTRACT

The present disclosure provides a sequencing method, which includes: performing capture treatment on an RNA molecule using a sequencing chip; performing reverse transcription treatment by using the captured RNA molecule as a template and Poly(dT) modified on the surface of the sequencing chip as a primer to synthesize 1^{st} cDNA, and then removing the RNA template; performing uridylation treatment on the 3′ terminus of the 1^{st} cDNA; performing ligation treatment on the uridylated 1^{st} cDNA and the 5′ terminus of a sequencing adapter; performing hybridization treatment on a product of the ligation treatment and a sequencing primer; performing extension treatment on the sequencing primer; performing a first cycle of sequencing extension on a product of the extension treatment; performing subsequent sequencing extension on a product of the first cycle of the sequencing extension. The method does not need a traditional library construction process, and can directly capture an RNA molecule and perform reverse transcription and sequencing in a sequencing chip, thereby fundamentally avoiding the high repeatability problem that may be caused by the PCR process of library construction.

### CROSS-REFERENCE TO RELATED APPLICATIONS

This application claims priority of Chinese patent application No. 202411764236.2 filed on Dec. 3, 2024, the entire contents of each of which are hereby incorporated by reference in its entirety.

### SEQUENCE LISTING

The instant application contains a Sequence Listing, which is submitted electronically in XML format and is hereby incorporated by reference in its entirety. The XML copy, created on Nov. 5, 2025, is named “2025-11-18-Sequence Listing-PI2023086US-20969-D018US00” and is 8,314-bytes in size.

### TECHNICAL FIELD

The present disclosure relates to the technical field of biology, particularly to a sequencing method, and more particularly to a method for direct sequencing of single-molecule 1st cDNA.

### BACKGROUND OF THE INVENTION

RNA (ribonucleic acid) is a ribonucleotide polymer with a variety of functions, which is widely involved in genetic expression activities of living organisms and can be broadly classified into coding RNA (coding RNA) and non-coding RNA (ncRNA) according to whether it encodes a protein or not. Coding RNA is mRNA (messenger RNA), which is a bridge for the transfer of genetic information from DNA to proteins. There are various types of ncRNAs, including rRNA, tRNA, lncRNA, circRNA, miRNA, siRNA, piRNA, and the like. Although ncRNAs do not encode proteins, they are involved in various levels of major life activities as indispensable organizers or regulatory molecules of cell functional systems and the like. Therefore, the research on RNA is of great significance for exploring the laws of life activities.

Over the past decade or so, RNA sequencing (RNA-seq) has gradually become an indispensable tool for transcriptome research. The wide use of RNA-seq has advanced the understanding of many biological dimensions, such as revealing the complexity of mRNA splicing and the mechanisms by which ncRNAs regulate gene expression. Currently, the most widely used RNA-seq is based on Illumina and similar platforms. According to different library construction methods, RNA sequencing is generally classified into whole transcriptome sequencing, mRNA sequencing, small RNA sequencing, circRNA sequencing, and the like. The main steps of the RNA sequencing generally include: RNA extraction, isolation and purification, RNA fragmentation (except for small RNA sequencing), reverse transcription to synthesize 1-stranded cDNA, synthesis of double-stranded DNA, ligation of an adapter (or the addition of an adapter in the process of the reverse transcription by using the template-switching activity of a reverse transcriptase), purification, PCR amplification, purification, quantification, on-line amplification into a cluster, and sequencing. The library construction process has many steps, is time-consuming, and has high reagent costs. Moreover, the PCR process results in high data duplication, which further affects the quantification of gene expression. Although the influence can be reduced by introducing unique molecular identifiers (UMIs) in library construction, it cannot prevent the generation of data duplication, nor can it prevent the influence of data duplication generated in the processes of clustering and sequencing.

Therefore, there is an urgent need to develop a novel RNA sequencing technology that can reduce the problem of high duplicate sequences and enable automated integration.

### SUMMARY

The present disclosure aims to solve, at least to some extent, at least one of the technical problems in the prior art. Based on this, in order to solve the problems described above, the present disclosure develops an innovative RNA sequencing scheme based on a single-molecule sequencing platform, i.e., terminal transferase-assisted uridine triphosphate (UTP)-mediated 1^{st} cDNA ligation sequencing. The method of the present disclosure solves the problem of high duplication caused in the process of PCR of library construction when RNA is sequenced on a next-generation sequencing (NGS) platform in principle. In addition, the method also simplifies the manual operation steps in the process of library construction, facilitating automated integration. Meanwhile, in the method, only one nucleotide filling step is required before sequencing, and the 3′ terminus of the cDNA does not need to be blocked with ddNTP. This improvement avoids sequencing errors and data waste caused by incomplete reactions, and improves the accuracy and efficiency of sequencing.

Therefore, in a first aspect of the present disclosure, provided is a sequencing method. According to the embodiments of the present disclosure, the method includes: performing capture treatment on an RNA molecule using a sequencing chip, where the surface of the sequencing chip is modified with Poly(dT), and the RNA molecule has a Poly(A) tail at the 3′ terminus; performing reverse transcription treatment by using the captured RNA molecule as a template and the Poly(dT) modified on the surface of the sequencing chip as a primer in the presence of dNTPs to synthesize 1^{st} cDNA, and then removing the captured RNA molecule; performing uridylation treatment on the 3′ terminus of the 1^{st} cDNA to obtain uridylated 1^{st} cDNA having an rU tail at the 3′ terminus; performing ligation treatment on the uridylated 1^{st} cDNA and the 5′ terminus of a sequencing adapter; performing hybridization treatment on a product of the ligation treatment and a sequencing primer, where the sequencing primer is complementarily paired with at least a part of the sequencing adapter; performing extension treatment on the sequencing primer by using the product of the ligation treatment as a template and the sequencing primer as a primer in the presence of dATP; performing a first cycle of sequencing extension on a product of the extension treatment by using at least one of dTTP, dGTP, and dCTP as a substrate; performing subsequent sequencing extension on a product of the first cycle of the sequencing extension by using at least one of dATP, dTTP, dGTP, and dCTP as a substrate.

The method of the present disclosure has unique advantages. Firstly, the method does not need to construct a DNA library by PCR amplification before sequencing, and can directly capture RNA molecules and perform reverse transcription and sequencing in a sequencing chip, thereby fundamentally avoiding the high duplication problem that may be caused by the traditional PCR process of library construction. This feature makes the method more efficient and faster. Secondly, in the method, Poly(dT) is designed on the surface of the sequencing chip as a probe to capture RNA, and a strategy of adding an rU tail to the 3′ terminus of the synthesized 1^{st} cDNA using uridine triphosphate (UTP) is adopted. This design enables efficient ligation of the 1^{st} cDNA to a sequencing adapter modified with a 3′ terminus blocking group, and simultaneously, allows convenient extension and completion of both the 1^{st} cDNA with the rU tail and ligated sequencing adapter, and the Poly(dT) with the rU tail and ligated sequencing adapter using dATP through a one-step filling reaction before sequencing, without the need to block the 3′ terminus of the 1st cDNA with ddNTP. This improvement not only simplifies the experimental steps, but also effectively avoids sequencing errors and data waste caused by incomplete reactions, thereby improving the accuracy and reliability of sequencing. In addition, the method simplifies the manual operation steps in the process of library construction, achieving automated integration. By reducing the intervention of manual operations, the complexity of the experiment is reduced, and the stability and consistency of the overall experiment are improved.

In a second aspect of the present disclosure, provided is a reagent kit. According to the embodiments of the present disclosure, the reagent kit includes a hybridization solution, a reverse transcription reagent, formamide, an rU tailing reagent, a proteinase K reagent (PK reagent), a ligation reagent, a sequencing primer reagent, a filling reagent, a first cleaning solution, and a second cleaning solution. The reagent kit of the present disclosure makes the whole sequencing process more efficient. By directly capturing RNA molecules and performing reverse transcription and sequencing in a sequencing chip, no traditional library construction process is required, and the time-consuming PCR step of library construction is omitted, thereby saving a large amount of time and experimental resources. In addition, the reagent kit simplifies the manual operation steps in the process of library construction. By providing an optimized reagent combination, it enables experimental personnel to perform experiments more easily and reduces potential operational errors. This simplified operation design helps to improve the consistency and stability of the experiment. In addition, the integration of the reagent kit with the automation platform is good. This allows the whole sequencing process to be conveniently automated, improving the efficiency and reproducibility of the experiment. The design of the reagent kit takes the requirement for automated integration into consideration, and provides the experimental personnel with a convenient choice.

In a third aspect of the present disclosure, provided is use of the reagent kit according to the second aspect in RNA sequencing. By using the reagent kit for RNA sequencing, RNA molecules can be directly sequenced without the need for traditional library construction processes, such as PCR of library construction. Therefore, the reagent kit can reduce the experimental cost and save a large amount of experimental time and resources for RNA sequencing, making the whole sequencing process more efficient.

Additional aspects and advantages of the present disclosure will in part be illustrated in the following description and become apparent from the following description, or may be learned by the implementation of the present disclosure.

### BRIEF DESCRIPTION OF THE DRAWINGS

The aforementioned and/or additional aspects and advantages of the present disclosure will become apparent and easily understood from the description of the embodiments with reference to the following drawings, in which:

FIG. 1 is a schematic diagram of the sequencing process according to examples of the present disclosure;

FIG. 2 is a diagram of the quantification results of 6 miRNAs according to the example of the present disclosure;

FIG. 3 is a diagram of the test results of the sequencing adapter ligation efficiency on the surface of the Flow Cell according to the example of the present disclosure.

### DETAILED DESCRIPTION OF THE INVENTION

Embodiments of the present disclosure are described in detail below. The embodiments described below are exemplary and are merely used to explain the present disclosure, and they should not be construed as limiting the present disclosure.

The term “at least one” means one or more, and “a plurality of” means two or more. “At least one” or similar expressions thereof refer to any combination of these items, including any combination of the singular or plural items. For example, “at least one of a, b, or c” or “at least one of a, b, and c” may each refer to: a, b, c, a-b (i.e., a and b), a-c, b-c, or a-b-c, where a, b, and c may be a single item or a plurality of items, respectively.

As used in the embodiments and the appended claims of the present disclosure, the singular forms “a”, “an”, and “the” are intended to include the plural forms as well, unless the context clearly indicates otherwise.

As used herein, the term “comprise”, “include”, or “contain” is open-ended, i.e., including what is meant by the present disclosure, but not excluding other aspects.

As used herein, the term “optionally” or “optional” generally means that the subsequently described event or condition may, but does not necessarily, occur, and that the description includes instances where the event or condition occurs and instances where the event or condition does not occur.

The terms “first”, “second”, “third”, and “fourth” are used for descriptive purposes only, are used for distinguishing purposes such as positions, objects, and the like from one another, and should not be understood as indicating or implying relative importance or implicitly indicating the number of technical features indicated. For example, without departing from the scope of the embodiments in the present disclosure, the terms “first” and “second” may be used interchangeably; for example, a first site may also be referred to as a second site, and correspondingly, a second site may be referred to as a first site. Therefore, features defined with “first”, “second”, “third”, and “fourth” may explicitly or implicitly include one or more of the features.

In the embodiments of the present disclosure, the term “sequencing” may also be referred to as “nucleic acid sequencing” or “gene sequencing”. The three are used interchangeably and refer to the determination of the type and sequence of bases or nucleotides (including nucleotide analogs) in a nucleic acid molecule. The sequencing involves the process of binding nucleotides to a template and collecting the corresponding signals emitted by the nucleotides (including analogs). The sequencing includes sequencing by synthesis (SBS) and/or sequencing by ligation (SBL), DNA sequencing and/or RNA sequencing, and long fragment sequencing and/or short fragment sequencing (the long fragment and short fragment are defined relatively; for example, nucleic acid molecules longer than 1 Kb, 2 Kb, 5 Kb, or 10 Kb may be referred to as long fragments, and nucleic acid molecules shorter than 1 Kb or 800 bp may be referred to as short fragments).

The sequencing generally involves multiple cycles of process to achieve the determination of the type and the sequential position of one base or nucleotide on one or more nucleic acid templates. The embodiments of the present disclosure refer to each cycle of the “process to achieve the determination of the type and the sequential position of one base or nucleotide on one or more nucleic acid templates” as one “cycle of sequencing”. The “cycle of sequencing”, also known as “sequencing cycle”, may be defined as the completion of one base extension of the four types of nucleotides/bases; in other words, one “cycle of sequencing” may be defined as the determination of the base or nucleotide type at any given position on the template. For sequencing platforms that achieve sequencing based on polymerization or ligation reactions, one cycle of sequencing includes the process of binding one of four types of nucleotides (including nucleotide analogs) to the nucleic acid template at a time according to the base complementary rule and acquiring the corresponding signals emitted. For platforms that achieve sequencing based on the polymerization reaction, a reaction system includes a nucleotide as a reaction substrate, a polymerase, and a nucleic acid template. A sequence fragment (a sequencing primer) binds to the nucleic acid template, and based on the base pairing rules and the principle of polymerization reaction, the nucleotide added as the reaction substrate is ligated to the sequencing primer under the catalysis of the polymerase to achieve the binding of the nucleotide to a specific position on the nucleic acid template. Generally, one cycle of sequencing may include one or more base extensions (repeats). For example, four types of nucleotides are sequentially added to the reaction system to each perform base extensions and corresponding acquisition of reaction signals, and one cycle of sequencing includes four base extensions; for another example, four types of nucleotides are added to the reaction system in any combinations (such as in pairs or in one-three combinations), the two combinations each perform base extensions and corresponding acquisition of reaction signals, and one cycle of sequencing includes two base extensions; for yet another example, four types of nucleotides are added simultaneously to the reaction system for base extension and acquisition of reaction signals, and one cycle of sequencing includes one base extension.

The term “template”, which may also be referred to as “nucleic acid template” or “sequencing template”, refers to a master nucleic acid molecule or master nucleic acid molecule fragment that binds to nucleotides or nucleotide analogs by multiple successive base extension cycles. The nucleic acid template may be the entire sequence of the nucleic acid molecule or a partial fragment of the nucleic acid molecule. The “template” may be a nucleic acid fragment used as a template in the extension reaction in which sequencing by synthesis is performed; since the base added in the extension reactions and the sequencing template satisfy the base pairing rules, the sequence of the sequencing template can be determined by determining the type of the base added in each extension cycle.

The term “primer”, also known as “probe”, refers to an oligonucleotide or a nucleic acid molecule that can hybridize to a target sequence of interest. In the embodiments, the primer serves as a substrate onto which nucleotides may be polymerized by a polymerase. For example, the primer may be used as a starting point for DNA or RNA synthesis. For example, a sequencing primer can hybridize to a synthesized nucleic acid template strand so as to trigger the synthesis of a new strand complementary to the synthesized nucleic acid template strand. The primer may include any combination of nucleotides or analogs thereof. In some examples, the primer is a single-stranded oligonucleotide or a polynucleotide.

The term “adapter”, also known as “linker” or “adaptor”, refers to an oligonucleotide fragment of a predetermined or known sequence, and the adapter is a single-stranded or double-stranded nucleic acid molecule. In commercially available mainstream sequencing platforms, the terminus of a nucleic acid fragment under test (also referred to as an insert fragment) from a sample is generally provided with a predetermined sequence (adapter) by processing, and the fragment under test is ligated or immobilized to a designated position of a reactor by using a primer or a probe (oligonucleotide strand) complementary to or binding to at least a part of the adapter (e.g., such primer or probe is immobilized on the designated surface of a flow cell or a chip). Based on the base complementary rules, at least a part of the sequence of the adapter can be used to design a primer/probe, and can be used as a binding site for a specific primer/probe.

“Ligation”, “immobilization”, and the like are to be construed in their broad sense; for example, they may refer to fixed ligation or reversible ligation, direct ligation, or indirect ligation via an intermediate medium, and may also be chemical bond ligation such as covalent ligation, chemical or physical adsorption, etc.

The term “nucleotide” refers to a molecule containing a sugar, at least one phosphate group, and in some examples, a nucleobase. The nucleotides include deoxyribonucleotides, modified deoxyribonucleotides, ribonucleotides, modified ribonucleotides, peptide nucleotides, modified peptide nucleotides, modified sugar-phosphate backbone nucleotides, and mixtures thereof. Examples of the nucleotide include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP). The nucleobase may be further modified. Exemplary modified nucleobases include inosine, xanthine, hypoxanthine, isocytosine, isoguanine, 2-aminopurine, 5-methylcytosine, 5-hydroxymethylcytosine, 2-aminoadenine, 6-methyladenine, 6-methylguanine, 2-propylguanine, 2-propyladenine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-substituted uracil, 4-thiouracil, 8-haloadenine or guanine, 8-aminoadenine or guanine, 8-thioladenine or guanine, 8-sulfanyladenine or guanine, 8-hydroxyadenine or guanine, 5-halouracil or cytosine, 7-methylguanine, 7-methyladenine, 8-azaguanine, 8-azaadenine, 7-deazaguanine, 7-deazaadenine, 3-deazaguanine, 3-deazaadenine, etc. As is known in the art, certain nucleotide analogs cannot be incorporated into polynucleotides, for example, nucleotide analogs such as adenosine 5′-phosphosulfate.

The term “polymerase” refers to an enzyme that has an active site for assembling polynucleotides by polymerizing nucleotides into polynucleotides. A polymerase may bind to the primed single-stranded target polynucleotide and may sequentially add nucleotides to the growing primer to form a “complementary copy” polynucleotide having a sequence that is complementary to that of the target polynucleotide. Another polymerase, or the same polymerase, may then form a copy of the target nucleotide by forming a complementary copy of the complementary copy polynucleotide. Any one of such copies may be referred to herein as an “amplicon”. DNA polymerase can bind to the target polynucleotide and then move downward along the target polynucleotide, sequentially adding nucleotides to the free hydroxyl group at the 3′ terminus of the growing polynucleotide strand (growing amplicon). DNA polymerases can synthesize complementary DNA molecules from a DNA template, and RNA polymerases can synthesize RNA molecules from a DNA template (transcription). Polymerases may use short RNA or DNA strands (primers) to start strand growth. Some polymerases can cause strand displacement upstream of the site where they add bases to the strand. Such polymerases may be referred to as strand-displacing, meaning they possess activity to remove the complementary strand from the template strand read by the polymerase. Exemplary polymerases with strand-displacing activity include, but are not limited to, a Bacillus stearothermophilus (Bst) polymerase, an exo-Klenow polymerase, or a large fragment of sequencing-grade T7 exo-polymerase. Some polymerases degrade the strand in front of them, effectively replacing the front strand with the growing back strand (5′ exonuclease activity). Some polymerases have an activity that degrades the strand behind them (3′ exonuclease activity). Some useful polymerases have been modified via mutation or other means to reduce or eliminate 3′ and/or 5′ exonuclease activity.

The term “locked nucleic acid” (abbreviated as LNA) refers to a synthetic nucleotide analog with a unique bicyclic structure. This structure is achieved by introducing a methylene bridge (or other types of bridging structures) between the 2′-OH and 4′-C of the ribose, thereby locking the ribose conformation into a specific shape, typically the 3′-endo conformation. The locking of this structure significantly enhances the binding affinity and thermal stability between LNA and its complementary DNA or RNA. Its structure is shown below:

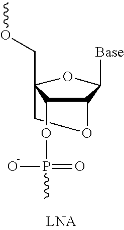

The “library” refers to a pool/collection of nucleic acid molecules containing multiple fragments of interest or under test, which are derived from the nucleic acid of the sample under test. Generally, the fragment of interest or under test are processed, for example, by adding a known sequence to one or both termini of the fragment of interest or under test, such as adding an adapter (sequencing adapter), to enable the processed fragment to ligate to or immobilize onto the chip, thereby being suitable for loading onto a sequencing platform for sequencing.

The present disclosure provides a sequencing method, a reagent kit, and use thereof, which will be described in detail below.

### Sequencing Method

The present disclosure provides a sequencing method, which includes: performing capture treatment on an RNA molecule using a sequencing chip, where the surface of the sequencing chip is modified with Poly(dT), and the RNA molecule has a Poly(A) tail at the 3′ terminus; performing reverse transcription treatment by using the captured RNA molecule as a template and the Poly(dT) modified on the surface of the sequencing chip as a primer in the presence of dNTPs to synthesize 1^{st} cDNA, and then removing the captured RNA molecule; performing uridylation treatment on the 3′ terminus of the 1^{st} cDNA to obtain uridylated 1^{st} cDNA having an rU tail at the 3′ terminus; performing ligation treatment on the 3′ terminus uridylated 1^{st} cDNA and the 5′ terminus of a sequencing adapter; performing hybridization treatment on a product of the ligation treatment and a sequencing primer, where the sequencing primer is complementarily paired with at least a part of the sequencing adapter; performing extension treatment on the sequencing primer by using the product of the ligation treatment as a template and the sequencing primer as a primer in the presence of dATP; performing a first cycle of sequencing extension on a product of the extension treatment by using at least one of dTTP, dGTP, and dCTP as a substrate; performing subsequent sequencing extension on a product of the first cycle of the sequencing extension by using at least one of dATP, dTTP, dGTP, and dCTP as a substrate.

The method of the present disclosure has unique advantages. Firstly, the method does not need to construct a DNA library by PCR amplification before sequencing, and can directly capture RNA molecules and perform reverse transcription and sequencing in a sequencing chip, thereby fundamentally avoiding the high duplication problem that may be caused by the traditional PCR process of library construction. This feature makes the method more efficient and faster. Secondly, the method adopts a strategy of adding an rU tail to the 3′ terminus of the synthesized 1^{st} cDNA using uridine triphosphate (UTP). This design enables efficient ligation of the 1^{st} cDNA to a sequencing adapter modified with a 3′ terminus blocking group and proceed in a reverse direction, and allows convenient extension and completion of both the 1^{st} cDNA with the rU tail and ligated sequencing adapter, and the Poly(dT) with the rU tail and ligated sequencing adapter using dATP through a one-step filling reaction before sequencing, without the need to block the 3′ terminus of the 1^{st} cDNA with ddNTP. This improvement not only simplifies the experimental steps, but also effectively avoids sequencing errors and data waste caused by incomplete reactions, thereby improving the accuracy and reliability of sequencing. In addition, the method simplifies the manual operation steps in the process of library construction, achieving automated integration. By reducing the intervention of manual operations, the complexity of the experiment is reduced, and the stability and consistency of the overall experiment are improved.

In some embodiments of the present disclosure, the sequencing method may further include at least one of the following additional technical features:

### Capture Treatment

In the embodiments of the present disclosure, the type of the RNA molecule is not specifically limited as long as it can be used for sequencing. Exemplarily, the RNA molecule may be selected from, but is not limited to, total RNA, mRNA, fragmented mRNA, miRNA, and cfRNA.

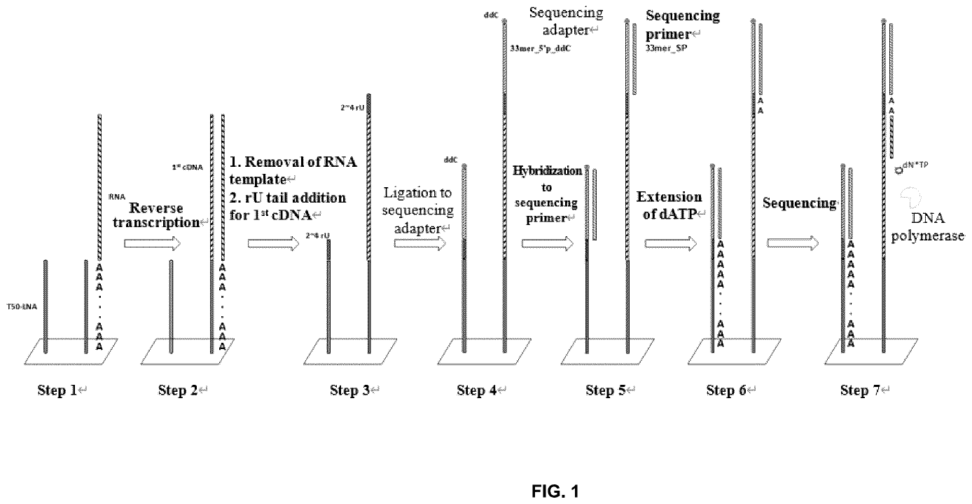

Referring to step 1 in FIG. 1, the surface of the sequencing chip is modified with a Poly(dT) probe. Unlike the case where the surface of the sequencing chip is modified with other probes or other polynucleotide Poly(dN) probes, mRNAs containing a Poly(A) tail can be directly captured after the surface of the sequencing chip is modified with the Poly(dT) probe in the embodiments of the present disclosure. Even if other types of RNA contain a Poly(A) tail, the Poly(A) tail can be easily added again using the Poly(A) polymerase. Since there is no adapter in the reaction system for adding the Poly(A) tail, the reaction product can be directly captured by the Poly(dT) on the surface of the sequencing chip without purification. In addition, since the number of probes on the surface of the sequencing chip is far greater than that of RNA, after the completion of reverse transcription, there are a large number of probes that have neither captured RNA nor generated 1^{st} cDNA. Even if treated with a single-stranded exonuclease (e.g., Exo I), these probes cannot be completely removed. The residual probes will also have an rU tail added, be ligated with sequencing adapters, and hybridize to sequencing primers in subsequent processes. Before sequencing, dATPs are used to achieve the completion of the rU tails, and at the same time, the Poly(dT) probes are subjected to blocking treatment. If non-polynucleotide probes are used, selective blocking cannot be performed. However, if other Poly(dN) probes besides Poly(dT) are used, the completion of the rU tails and the blocking of the Poly(dT) probes need to be performed separately. Specifically, it is necessary to additionally introduce complementary dNTPs for blocking, which increases the complexity of the operation process.

In some embodiments of the present disclosure, the 3′ terminus of the RNA molecule carries a Poly(A) tail. When the RNA molecule is selected from fragmented mRNA, miRNA, and cfRNA, an RNA molecule having a Poly(A) tail at the 3′ terminus is obtained by pre-adding a Poly(A) tail to the 3′ terminus of the RNA target molecule.

In the embodiments of the present disclosure, the Poly(A) tail may be added to the 3′ terminus of the RNA target molecule by ligation reaction or polymerization extension.

In one embodiment, the Poly(A) tail is added to the 3′ terminus of the RNA target molecule by a polymerization extension method, which can be used to perform 3′ terminal Poly(A) tailing treatment on an RNA target molecule such as a fragmented mRNA molecule. In this case, the addition of the Poly(A) tail can be achieved using a Poly(A) tailing reagent kit. In some embodiments, a Poly(A) tailing reagent kit includes essentially a Poly(A) polymerase, a buffer, and ATP. The polymerase can be replaced with any enzyme capable of adding nucleotides to the 3′ terminus of an RNA target molecule in a non-template-dependent manner. The length of the Poly(A) tail can be set by regulating the molar ratio of ATP to the RNA target molecule. Exemplarily, by adjusting the molar ratio of ATP to the RNA target molecule to 100:1 to 500:1, typically 20-300 A bases can be added to the 3′ terminus of the RNA target molecule to form a Poly(A) tail. Exemplarily, a Poly(A) tailing reagent kit includes a Poly(A) polymerase reaction buffer, ATP, and a Poly(A) polymerase, and through the reagent kit, a Poly(A) tail can be formed at the 3′ terminus of an RNA molecule by polymerization extension. In some embodiments, the reagent used in the process of the 3′ terminal Poly(A) tailing treatment further includes nuclease-free ddH₂O, a molecular crowding agent, an RNase inhibitor, and T4-PNK.

In some embodiments, the molecular crowding agent is a macromolecular polymer selected from at least one of PEG, PVP, and dextran. In some embodiments, a mean molecular weight range of the PEG is 4000-100000. Exemplarily, the PEG is selected from PEG4000, PEG6000, PEG8000, PEG10000, PEG20000, PEG35000, PEG80000, and PEG100000. In some embodiments, a mean molecular weight range of the PVP is 10000-60000. Exemplarily, the PVP is selected from PVP10000, PVP24000, PVP40000, and PVP55000. In some embodiments, a mean molecular weight range of the dextran is 10000-70000. Exemplarily, the dextran is selected from dextran 10000, dextran 20000, dextran 40000, and dextran 70000.

In order to improve the hybridization stability of the RNA to the Poly(dT) probe on the surface of the sequencing chip, in some embodiments of the present disclosure, a locked nucleic acid (LNA) modification is introduced into the Poly(dT). The locked nucleic acid modification can increase the Tm (DNA melting temperature) value of nucleic acid hybridization and promote the stability of the hybridized nucleic acid double-stranded structure; meanwhile, the modification increases the hybridization temperature during probe capture and promote the formation of a longer and more stable hybridization region between the Poly(A) tail at the 3′ terminus of the RNA molecule and the Poly(dT) into which the locked nucleic acid is introduced. Considering the sequence length of Poly(dT) as a capture probe, 1-5 locked nucleic acids are introduced into Poly(dT) for modification. Exemplarily, the number of locked nucleic acids introduced into Poly(dT) is 1, 2, 3, 4, or 5, i.e., the Poly(dT) includes 1-5 dTTPs having a locked nucleic acid modification.

In order to make the hybridization of RNA to Poly(dT) on the surface of the sequencing chip efficient, the capture treatment was performed in a hybridization solution. In one example, the hybridization solution includes sodium chloride, sodium citrate, sodium dodecyl sulfate (SDS), dextran sulfate, and formamide. In this case, the capture of the RNA molecule by the Poly(dT) probe with locked nucleic acids can be promoted under the action of the hybridization solution, facilitating the binding of the RNA onto the surface of the sequencing chip.

In some embodiments of the present disclosure, a final concentration of the sodium chloride in the hybridization solution is 680 mmol/L-750 mmol/L. Exemplarily, the final concentration may be, but is not limited to, any one of or a range between any two of 680 mmol/L, 685 mmol/L, 690 mmol/L, 695 mmol/L, 700 mmol/L, 705 mmol/L, 710 mmol/L, 715 mmol/L, 720 mmol/L, 725 mmol/L, 730 mmol/L, 735 mmol/L, 740 mmol/L, 745 mmol/L, and 750 mmol/L.

In some embodiments of the present disclosure, a final concentration of the sodium citrate in the hybridization solution is 68 mmol/L-75 mmol/L. Exemplarily, the final concentration may be, but is not limited to, any one of or a range between any two of 68 mmol/L, 68.5 mmol/L, 69 mmol/L, 69.5 mmol/L, 70 mmol/L, 70.5 mmol/L, 71 mmol/L, 71.5 mmol/L, 72 mmol/L, 72.5 mmol/L, 73 mmol/L, 73.5 mmol/L, 74 mmol/L, 74.5 mmol/L, and 75 mmol/L.

In some embodiments of the present disclosure, a volume fraction (volume ratio, v/v) of the sodium dodecyl sulfate in the hybridization solution is 0.05%-0.1%. Exemplarily, the volume fraction may be, but is not limited to, any one of or a range between any two of 0.05%, 0.06%, 0.07%, 0.08%, 0.09%, and 0.1%.

In some embodiments of the present disclosure, a volume fraction of the dextran sulfate in the hybridization solution is 14%-20%. Exemplarily, the volume fraction may be, but is not limited to, any one of or a range between any two of 14%, 15%, 16%, 17%, 18%, 19%, and 20%.

In some embodiments of the present disclosure, a volume fraction of the formamide in the hybridization solution is 45%-50%. Exemplarily, the volume fraction may be, but is not limited to, any one of or a range between any two of 45%, 46%, 47%, 48%, 49%, and 50%.

In some embodiments of the present disclosure, the RNA molecule is selected from fragmented mRNA, miRNA, and cfRNA, and the RNA molecule having the Poly(A) tail at the 3′ terminus is provided in a form dissolved in water, i.e., the RNA molecule having the Poly(A) tail at the 3′ terminus is dissolved in water to form an RNA molecule solution. In some embodiments, a concentration of the RNA molecule in the RNA molecule solution is 0.5 nmol/L-2 nmol/L, for example, may be any one of or a range between any two of 0.5 nmol/L, 1 nmol/L, 1.5 nmol/L, and 2 nmol/L.

In some embodiments of the present disclosure, the RNA molecule is selected from total RNA, and a mass-volume ratio of the RNA molecule to the hybridization solution is 2-20 ng/μL. Exemplarily, the mass-volume ratio may be, but is not limited to, any one of or a range between any two of 2 ng/μL, 4 ng/μL, 6 ng/μL, 8 ng/μL, 10 ng/μL, 12 ng/μL, 14 ng/μL, 16 ng/μL, 18 ng/μL, and 20 ng/μL.

In some embodiments of the present disclosure, a volume ratio of the RNA molecule solution to the hybridization solution is 3:5.

In some embodiments of the present disclosure, the capture treatment is performed at 30-45° C. for 20-40 min. In this case, the capture efficiency of Poly(dT) for RNA molecules can be improved.

Exemplarily, the capture treatment is performed at 30° C., 32° C., 35° C., 37° C., 39° C., 41° C., 43° C., or 45° C. for 20 min, 22 min, 24 min, 26 min, 28 min, 30 min, 32 min, 34 min, 36 min, 38 min, or 40 min.

### Reverse Transcription Treatment

In this step, as shown in step 2 in FIG. 1, reverse transcription treatment is performed by using the captured RNA molecule as a template and Poly(dT) modified on the surface of the sequencing chip as a primer in the presence of dNTP to obtain 1^{st} cDNA.

In order to increase the yield of the 1^{st} cDNA product, a special reverse transcription reagent is formulated in the embodiments of the present disclosure by adding betaine, Ficoll-400, poloxamer 188, or Triton X-100 to a reaction buffer system. In exemplary embodiments, the reverse transcription reagent includes a reverse transcriptase, dNTP, an RNase inhibitor, poloxamer 188, tris(hydroxymethyl)aminomethane hydrochloride (Tris-HCl), potassium chloride, magnesium chloride, dithiothreitol, and nuclease-free water. In this case, by performing a reverse transcription treatment in the reverse transcription reagent provided by the embodiments of the present disclosure, cDNA can be synthesized rapidly and accurately, significantly increasing the yield of the cDNA product.

In some embodiments of the present disclosure, a concentration of the reverse transcriptase in the reverse transcription reagent is 5 U/μL-15 U/μL. Exemplarily, the concentration may be, but is not limited to, any one of or a range between any two of 5 U/μL, 6 U/μL, 7 U/μL, 8 U/μL, 9 U/μL, 10 U/μL, 11 U/μL, 12 U/μL, 13 U/μL, 14 U/μL, and 15 U/μL.

In some embodiments of the present disclosure, a final concentration of the dNTP in the reverse transcription reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In some embodiments of the present disclosure, a final concentration of the RNase inhibitor in the reverse transcription reagent is 0.1 U/μL-1 U/μL. Exemplarily, the final concentration may be, but is not limited to, any one of or a range between any two of 0.1 U/μL, 0.2 U/μL, 0.3 U/μL, 0.4 U/μL, 0.5 U/μL, 0.6 U/μL, 0.7 U/μL, 0.8 U/μL, 0.9 U/μL, and 1 U/μL.

In some embodiments of the present disclosure, a volume fraction of the poloxamer 188 in the reverse transcription reagent is 5%-15%. Exemplarily, the volume fraction may be, but is not limited to, any one of or a range between any two of 5%, 6%, 7%, 8%, 9%, 10%, 11%, 12%, 13%, 14%, and 15%.

In some embodiments of the present disclosure, a final concentration of the tris(hydroxymethyl)aminomethane hydrochloride in the reverse transcription reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the potassium chloride in the reverse transcription reagent is 70 mmol/L-80 mmol/L. Exemplarily, the final concentration may be, but is not limited to, any one of or a range between any two of 71 mmol/L, 72 mmol/L, 73 mmol/L, 74 mmol/L, 75 mmol/L, 76 mmol/L, 77 mmol/L, 78 mmol/L, 79 mmol/L, and 80 mmol/L.

In some embodiments of the present disclosure, a final concentration of the magnesium chloride in the reverse transcription reagent is 1 mmol/L-5 mmol/L. Exemplarily, the final concentration may be, but is not limited to, any one of or a range between any two of 1 mmol/L, 2 mmol/L, 3 mmol/L, 4 mmol/L, and 5 mmol/L.

In some embodiments of the present disclosure, a final concentration of the dithiothreitol in the reverse transcription reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In order to improve the reaction efficiency and the product yield, in the process of the reverse transcription treatment, the reverse transcription reagent is introduced into the sequencing chip in batches, allowing the reverse transcription reagent to be in contact with RNA molecules. This ensures a more thorough reverse transcription reaction and improves the reverse transcription effect. In some embodiments, the reverse transcription reagent is introduced in 2-5 batches. Exemplarily, the reverse transcription reagent is introduced in 2 batches, 3 batches, 4 batches, or 5 batches.

In some embodiments of the present disclosure, in the process of the reverse transcription treatment, the reverse transcription reagent is introduced in 3 batches to facilitate the progress of the reverse transcription and increase the yield of 1^{st} cDNA, a product of the reverse transcription.

The amount of the reverse transcription reagent introduced in batches is not specifically limited in the present disclosure. In one embodiment, the amount of the reverse transcription reagent introduced in the first batch can at least replace the reagent in the pipeline and the sequencing chip or fill the pipeline and the sequencing chip, and the amount of the reverse transcription reagent introduced in the second batch and the amount of the reverse transcription reagent introduced in the third batch can at least replace the reverse transcription reagent introduced into the sequencing chip in the previous batch. The more the reverse transcription reagent is introduced, the higher the replacement ratio and reaction efficiency will be. However, this also leads to increased reagent consumption and higher costs.

In some embodiments of the present disclosure, after each introduction of the reverse transcription reagent, the reverse transcription treatment is performed at 41° C.-45° C. for 10-30 min. Therefore, thorough progression of the reverse transcription treatment can be promoted, and the synthesis of the reverse transcription product, 1^{st} cDNA, can be enhanced.

Exemplarily, the reverse transcription treatment is performed at 41° C., 42° C., 43° C., 44° C., or 45° C. for 10 min, 12 min, 14 min, 16 min, 18 min, 20 min, 22 min, 24 min, 26 min, 28 min, or 30 min.

In some embodiments, after the reverse transcription is completed, the sequencing chip is rinsed with a first cleaning solution to remove the residual reverse transcription reagent on the surface. Exemplarily, the first cleaning solution includes sodium chloride, sodium citrate, 4-hydroxyethylpiperazine ethanesulfonic acid, and sodium dodecyl sulfate. The cleaning solution can effectively remove the residual reverse transcription reagent on the surface of the sequencing chip without affecting the stability of 1^{st} cDNA.

### Removal of RNA Template

Referring to step 2 in FIG. 1, after the synthesis of the product 1^{st} cDNA through the reverse transcription treatment, a 1^{st} cDNA/RNA double-stranded structure is formed, and in order to remove the RNA from the 1^{st} cDNA/RNA duplex, a variety of methods can be used. For example, by increasing the temperature of the chip or altering the buffer conditions (exemplarily, using formamide or a 0.1 M sodium hydroxide solution), the 1^{st} cDNA/RNA duplex is destabilized, leading to denaturation. Alternatively, RNase H is used to specifically remove the RNA in the 1st cDNA/RNA duplex. Subsequently, a suitable buffer is used to rinse the chip surface, removing the RNA while retaining the 1^{st} cDNA that is obtained via extension from the Poly(dT-LNA) (Poly(dT) containing a locked nucleic acid modification) covalently ligated to the chip surface, as shown in step 3 in FIG. 1.

In one embodiment of the present disclosure, formamide is used to remove the RNA template, thereby removing the RNA from the 1st cDNA/RNA duplex and retaining only the product 1^{st} cDNA of the reverse transcription treatment for subsequent operations. In some embodiments, the RNA template is removed using formamide at a temperature of 50° C.-60° C. Specifically, the RNA template may be removed at a temperature of 50° C., 51° C., 52° C., 53° C., 54° C., 55° C., 56° C., 57° C., 58° C., 59° C., 60° C., or the like. In some embodiments, after formamide is mixed with a first cleaning solution, the RNA template is removed, and thus the RNA in the 1st cDNA/RNA duplex is removed.

In some embodiments, after the removal of the RNA template is completed, the sequencing chip is rinsed sequentially with a first cleaning solution and a second cleaning solution to remove the residual formamide on the surface. Exemplarily, the first cleaning solution includes sodium chloride, sodium citrate, 4-hydroxyethylpiperazine ethanesulfonic acid, and sodium dodecyl sulfate. Exemplarily, the second cleaning solution includes sodium chloride and sodium citrate.

### Uridylation Treatment

In order to effectively improve the ligation efficiency of 1st cDNA and a sequencing adapter, particularly an ssDNA adapter or an RNA adapter, in the embodiments of the present disclosure, the 3′ terminus of the obtained 1^{st} cDNA is subjected to uridylation treatment using a terminal transferase (TdT) and uridine triphosphate (UTP) to obtain uridylated 1st cDNA having an rU(ribo-uridine) tail at the 3′ terminus. In this embodiment, the addition of the rU tail can promote the ligation between the 1^{st} cDNA and the sequencing adapter in the following steps, while the terminal transferase (TdT) can achieve efficient extension of the uridine triphosphate (UTP) at the 3′ terminus of the 1^{st} cDNA, with the reaction automatically terminating after typically adding 2-4 bases or uridine triphosphates (UTPs) to limit the base length of the resulting rU tail. It should be understood that during the addition of the rU tail to the 3′ terminus of the 1^{st} cDNA, the redundant Poly(dT) on the surface of the sequencing chip also simultaneously acquires an equivalent quantity of UTP at its 3′ terminus, forming an rU tail. In some embodiments, as shown in step 3 in FIG. 1, 2-4 uridine triphosphates (UTPs) are added to the 3′ terminus of the 1^{st} cDNA to form an rU tail. Another reason for using UTP to add an rU tail at the 3′ terminus of st cDNA is that: after an rU tail is added at the 3′ terminus, during the sequencing of 1^{st} cDNA, dATPs are used in a one-step filling reaction to complete the base complementation for the rU added to the 3′ terminus of the 1^{st} cDNA, and simultaneously, to complete the base complementation for the Poly(dT) on the surface of the sequencing chip that is not hybridized by RNA, thereby preventing the rU tail and Poly(dT) from introducing sequencing noise during 1^{st} cDNA sequencing. In contrast, if ATP, CTP, or GTP is used to form a tail at the 3′ terminus of the 1^{st} cDNA, a two-step filling reaction is required for completion, increasing both the reaction time and cost. Therefore, the strategy of using UTP to add an rU tail significantly improves the ligation efficiency and simplifies the operation, making it simpler and more efficient.

In some embodiments of the present disclosure, the uridylation treatment is performed in an rU tailing reagent. Specifically, the rU tailing reagent includes TdT, UTP, tris(hydroxymethyl)aminomethane acetate (Tris-acetate), potassium acetate, magnesium acetate, CoCl₂, BSA (bovine serum albumin), and nuclease-free water.

In some embodiments of the present disclosure, a final concentration of the TdT in the rU tailing reagent is 0.1 U/μL-0.3 U/μL. Exemplarily, the final concentration may be, but is not limited to, any one of or a range between any two of 0.1 U/μL, 0.2 U/μL, and 0.3 U/μL.

In some embodiments of the present disclosure, a final concentration of the UTP in the rU tailing reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In some embodiments of the present disclosure, a final concentration of the tris(hydroxymethyl)aminomethane acetate in the rU tailing reagent is 10 mmol/L-30 mmol/L. Exemplarily, the final concentration may be, but is not limited to, any one of or a range between any two of 10 mmol/L, 12 mmol/L, 14 mmol/L, 16 mmol/L, 18 mmol/L, 20 mmol/L, 22 mmol/L, 24 mmol/L, 26 mmol/L, 28 mmol/L, and 30 mmol/L.

In some embodiments of the present disclosure, a final concentration of the potassium acetate in the rU tailing reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the magnesium acetate in the rU tailing reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In some embodiments of the present disclosure, a final concentration of the CoCl₂ in the rU tailing reagent is 0.1 mmol/L-0.5 mmol/L. Exemplarily, the final concentration may be, but is not limited to, any one of or a range between any two of 0.1 mmol/L, 0.2 mmol/L, 0.3 mmol/L, 0.4 mmol/L, and 0.5 mmol/L.

In some embodiments of the present disclosure, a final concentration of the BSA in the rU tailing reagent is 0.05 mg/mL-0.15 mg/mL. Exemplarily, the final concentration may be, but is not limited to, any one of or a range between any two of 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, and 0.15 mg/mL.

In order to improve the efficiency of adding the rU tail to the 3′ terminus of the 1^{st} cDNA, in the process of the uridylation treatment, the rU tailing reagent is introduced onto the surface of the sequencing chip in 2-5 batches. Thus, the reaction efficiency and the quality of the product can be improved. Exemplarily, the rU tailing reagent is introduced in 2 batches, 3 batches, 4 batches, or 5 batches.

In some embodiments of the present disclosure, in the process of the uridylation treatment, the rU tailing reagent is introduced in 3 batches.

The amount of the rU tailing reagent introduced in batches is not specifically limited in the present disclosure. In one embodiment, the amount of the rU tailing reagent introduced in the first batch can at least replace the reagent in the pipeline and the sequencing chip or fill the pipeline and the sequencing chip, and the amount of the rU tailing reagent introduced in the second batch and the amount of the rU tailing reagent introduced in the third batch can at least replace the rU tailing reagent introduced into the sequencing chip in the previous batch. The more the rU tailing reagent is introduced, the higher the replacement ratio and reaction efficiency will be. However, this also leads to increased reagent consumption and higher costs.

In some embodiments of the present disclosure, after each introduction of the rU tailing reagent, the uridylation treatment is performed at 35-40° C. for 10-30 min. Therefore, thorough progression of the uridylation treatment can be promoted, and the reaction rate can be increased.

Exemplarily, the uridylation treatment is performed at 35° C., 36° C., 37° C., 38° C., 39° C., or 40° C. for 10 min, 12 min, 14 min, 16 min, 18 min, 20 min, 22 min, 24 min, 26 min, 28 min, or 30 min.

In some embodiments, after the uridylation treatment is completed, the sequencing chip is rinsed with a first cleaning solution to remove the residual rU tailing reagent on the surface.

During the experiment, the inventors found that the TdT used in the process of the uridylation treatment adsorbs to the surface of the sequencing chip, and in the subsequent sequencing, a large number of fluorescent bases can be adsorbed, thereby causing the failure to correctly identify the sequencing base signals. Based on this, the inventors performed a washing treatment after the uridylation treatment, mainly to remove TdT from the surface of the sequencing chip and prevent it from affecting the sequencing results.

The inventors found that TdT cannot be removed or completely removed by using a common cleaning solution or ddH₂O. Therefore, a special cleaning solution, i.e., a PK reagent, is formulated in the present disclosure, and the PK reagent includes proteinase K, tris(hydroxymethyl)aminomethane hydrochloride, sodium chloride, sodium dodecyl sulfonate (SDSO), ethylenediaminetetraacetic acid disodium (disodium EDTA, EDTA disodium, EDTA-2Na), and nuclease-free water. The main role is proteinase K, which can hydrolyze TdT, thereby enabling the complete removal of TdT on the surface of the sequencing chip.

A concentration of the proteinase K in the PK reagent is 0.05 mg/mL-0.15 mg/mL. Exemplarily, the concentration may be, but is not limited to, any one of or a range between any two of 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, and 0.15 mg/mL. Therefore, TdT on the surface of the sequencing chip can be completely removed, thereby ensuring the accuracy of subsequent sequencing results.

In some embodiments of the present disclosure, a final concentration of the tris(hydroxymethyl)aminomethane hydrochloride in the PK reagent is 95 mmol/L-105 mmol/L. Exemplarily, the final concentration may be, but is not limited to, any one of or a range between any two of 95 mmol/L, 96 mmol/L, 97 mmol/L, 98 mmol/L, 99 mmol/L, 100 mmol/L, 101 mmol/L, 102 mmol/L, 103 mmol/L, 104 mmol/L, and 105 mmol/L.

In some embodiments of the present disclosure, a final concentration of the sodium chloride in the PK reagent is 195 mmol/L-205 mmol/L. Exemplarily, the final concentration may be, but is not limited to, any one of or a range between any two of 195 mmol/L, 196 mmol/L, 197 mmol/L, 198 mmol/L, 199 mmol/L, 200 mmol/L, 201 mmol/L, 202 mmol/L, 203 mmol/L, 204 mmol/L, and 205 mmol/L.

In some embodiments of the present disclosure, a volume fraction of the sodium dodecyl sulfonate in the PK reagent is 0.05%-0.15%. Exemplarily, the volume fraction may be, but is not limited to, any one of or a range between any two of 0.05%, 0.06%, 0.07%, 0.08%, 0.09%, 0.1%, 0.11%, 0.12%, 0.13%, 0.14%, and 0.15%.

In some embodiments of the present disclosure, a concentration of the EDTA disodium in the PK reagent is 5 mmol/L-15 mmol/L. Exemplarily, the concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In order to completely remove TdT on the surface of the sequencing chip, in the process of the washing treatment, the PK reagent is introduced onto the surface of the sequencing chip in 2-5 batches. Exemplarily, the PK reagent may be introduced in 2 batches, 3 batches, 4 batches, or 5 batches.

The amount of the PK reagent introduced in batches is not specifically limited in the present disclosure. In one embodiment, the amount of the PK reagent introduced in the first batch can at least replace the reagent in the pipeline and the sequencing chip or fill the pipeline and the sequencing chip, and the amount of the PK reagent introduced in the second batch and the amount of the PK reagent introduced in the third batch can at least replace the PK reagent introduced into the sequencing chip in the previous batch. The more the PK reagent is introduced, the higher the replacement ratio and reaction efficiency will be. However, this also leads to increased reagent consumption and higher costs.

In some embodiments of the present disclosure, after each introduction of the PK reagent, the washing treatment is performed at 50-60° C. for 3-8 min.

Exemplarily, the washing treatment is performed at 50° C., 51° C., 52° C., 53° C., 54° C., 55° C., 56° C., 57° C., 58° C., 59° C., or 60° C. for 3 min, 4 min, 5 min, 6 min, 7 min, or 8 min. Therefore, TdT on the surface of the sequencing chip can be completely removed, thereby ensuring the accuracy of the sequencing results.

In some embodiments, after the cleaning with the PK reagent is completed, the sequencing chip is rinsed sequentially with a first cleaning solution and a second cleaning solution to remove the residual PK reagent on the surface.

### Ligation Treatment

In this step, as shown in step 4 in FIG. 1, the 5′ terminus of a sequencing adapter consisting of a specific sequence is ligated to the 3′ terminus of the 1^{st} cDNA added with the rU tail (i.e., the 3′ terminus of the uridylated 1^{st} cDNA) using a suitable RNA ligase. The sequencing adapter used may be ssDNA or RNA. ssDNA offers better stability and is more cost-effective than RNA. The 5′ terminus of the sequencing adapter features a phosphorylation modification or an adenylation modification. While the optimized ligation efficiency of the phosphorylated version is comparable to that of the adenylated version, the phosphorylated version is more cost-effective than the adenylated version. Furthermore, the 3′ terminus of the sequencing adapter may be modified with a blocking group, generally a nucleotide terminator lacking 3′-OH, such as: 2′,3′-dideoxynucleotide, 3′-aminonucleotide, 3′-deoxynucleotide, and 3′-azidonucleotide. The blocking group functions to prevent self-ligation, cyclization, and repeated ligation of the adapter sequence.

The ligation treatment reaction catalyzed by an RNA ligase is divided into three steps. First, the RNA ligase reacts with ATP to form an adenylated enzyme intermediate, releasing pyrophosphate (PPi). Then, the formed adenylated enzyme intermediate transfers its adenosine monophosphate (AMP) group to the 5′ phosphate of ssRNA or ssDNA, generating an activated 5′-adenylated intermediate product, 5′AppRNA or single-stranded 5′AppDNA donor. Finally, the RNA ligase catalyzes the substitution of the 5′-AMP of the donor with the 3′-OH of the product of the uridylation treatment to form a phosphodiester bond and complete the ligation reaction. When the ATP concentration in the system is high, the RNA ligase, which has lost AMP during the formation of the 5′AppRNA or single-stranded 5′AppDNA donor in the second step, readily reacts with ATP again to re-form the adenylated enzyme intermediate, thereby preventing it from catalyzing the subsequent ligation reaction. When the ATP concentration is low, the first two steps of the reaction are impeded. Therefore, optimizing the relative molar ratios among the ATP, the RNA ligase, and the 5′ phosphate donor in the reaction system is the key to improving the ligation efficiency. Through research, the inventors found that the ligation efficiency is up to about 90% when a molar ratio of ATP to RNA ligase to 5′ phosphate donor is 5:1:1 to 50:1:1.

In order to reduce research costs without compromising ligation efficiency, a special ligation reagent is formulated in the present disclosure, and the ligation reagent includes T4 RNA ligase 1, a sequencing adapter, a molecular crowding agent, ATP, tris(hydroxymethyl)aminomethane hydrochloride, magnesium chloride, dithiothreitol, and nuclease-free water. The use of expensive T4 RNA ligase 2 and 5′ adenylation modification is avoided for the ligation reagent. Instead, through the use of T4 RNA ligase 1 and a sequencing adapter featuring a phosphorylation modification at the 5′ terminus and a ddC (dideoxycytosine, for 3′ terminus blocking) modification at the 3′ terminus, research costs are reduced without compromising the ligation efficiency.

In some embodiments of the present disclosure, a final concentration of the T4 RNA ligase 1 in the ligation reagent is 0.1 U/μL-1 U/μL. Exemplarily, the final concentration may be, but is not limited to, any one of or a range between any two of 0.1 U/μL, 0.2 U/μL, 0.3 U/μL, 0.4 U/μL, 0.5 U/μL, 0.6 U/μL, 0.7 U/μL, 0.8 U/μL, 0.9 U/μL, and 1 U/μL.

In some embodiments of the present disclosure, the 5′ terminus of the sequencing adapter has a phosphorylation modification or an adenylation modification.

In some embodiments of the present disclosure, the 3′ terminus of the sequencing adapter has a blocking group modification, and the blocking group functions to prevent self-ligation, cyclization, and repeated ligation of the adapter sequence, thereby avoiding self-cyclization of the RNA molecule. The blocking group is selected from a nucleotide terminator lacking 3′-OH on the ribose, and exemplarily, the blocking group may be 2′,3′-dideoxynucleotide, 3′-aminonucleotide, or 3′-deoxynucleotide.

In some embodiments of the present disclosure, a final concentration of the sequencing adapter in the ligation reagent is 0.5 μmol/L-1.5 μmol/L. Exemplarily, the final concentration may be, but is not limited to, any one of or a range between any two of 0.5 μmol/L, 0.6 μmol/L, 0.7 μmol/L, 0.8 μmol/L, 0.9 μmol/L, 1 μmol/L, 1.1 μmol/L, 1.2 μmol/L, 1.3 μmol/L, 1.4 μmol/L, and 1.5 μmol/L.

In some embodiments of the present disclosure, the sequencing adapter has the nucleotide sequence set forth in SEQ ID NO: 1.

(SEQ ID NO: 1)
5′p-TGGACATCTCGGGTGCCAAGGAACTCACGTCAC-ddC

In some embodiments of the present disclosure, a volume fraction of the molecular crowding agent in the ligation reagent is 15%-25%. Exemplarily, the volume fraction may be, but is not limited to, any one of or a range between any two of 15%, 16%, 17%, 18%, 19%, 20%, 21%, 22%, 23%, 24%, and 25%.

In some embodiments of the present disclosure, a final concentration of the ATP in the ligation reagent is 0.05 mmol/L-0.15 mmol/L. Exemplarily, the final concentration may be, but is not limited to, any one of or a range between any two of 0.05 mmol/L, 0.06 mmol/L, 0.07 mmol/L, 0.08 mmol/L, 0.09 mmol/L, 0.1 mmol/L, 0.11 mmol/L, 0.12 mmol/L, 0.13 mmol/L, 0.14 mmol/L, and 0.15 mmol/L.

In some embodiments of the present disclosure, a final concentration of the tris(hydroxymethyl)aminomethane hydrochloride in the ligation reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the magnesium chloride in the ligation reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In some embodiments of the present disclosure, a final concentration of the dithiothreitol in the ligation reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In the process of the ligation treatment, the ligation reagent is introduced onto the surface of the sequencing chip in 2-5 batches, so that the ligation efficiency and product quality can be improved, and the ligation quality of the sequencing adapter and the accuracy of the sequencing results can be ensured. Exemplarily, the ligation reagent may be introduced in 2 batches, 3 batches, 4 batches, or 5 batches.

The amount of the ligation reagent introduced in batches is not specifically limited in the present disclosure, as long as the amount of the ligation reagent introduced in the first batch can at least replace the reagent in the pipeline and the sequencing chip or fill the pipeline and the sequencing chip, and the amount of the ligation reagent introduced in the second batch and the amount of the ligation reagent introduced in the third batch can at least replace the ligation reagent introduced into the sequencing chip in the previous batch. The more the ligation reagent is introduced, the higher the replacement ratio and reaction efficiency will be. However, this also leads to increased reagent consumption and higher costs.

In some embodiments of the present disclosure, after each introduction of the ligation reagent, the ligation treatment is performed at 20° C.-30° C. for 20-40 min. Therefore, the successful ligation of the sequencing adapter can be ensured, thereby guaranteeing the accuracy of the sequencing results.

Exemplarily, the ligation treatment is performed at 20° C., 22° C., 24° C., 25° C., 27° C., 29° C., or 30° C. for 20 min, 22 min, 24 min, 26 min, 28 min, 30 min, 32 min, 34 min, 36 min, 38 min, or 40 min.

In some embodiments, after the ligation treatment is completed, the sequencing chip is rinsed with a first cleaning solution to remove the residual sequencing primer reagent on the surface.

### Hybridization Treatment

After the ligation treatment is completed, referring to step 5 in FIG. 1, an ssDNA sequence that is reversely complementary to the sequencing adapter sequence is hybridized to serve as the sequencing primer.

In this step, a hybridization treatment is performed using a sequencing primer reagent. The sequencing primer reagent includes a sequencing primer, sodium chloride, sodium citrate, sodium dodecyl sulfate, dextran sulfate, formamide, and nuclease-free water. Exemplarily, the sequencing primer has the nucleotide sequence set forth in SEQ ID NO: 2.

(SEQ ID NO: 2)
GTGACGTGAGTTCCTTGGCACCCGAGATGTCCA

In some embodiments of the present disclosure, a final concentration of the sequencing primer in the sequencing primer reagent is 0.05 μmol/L-0.15 μmol/L. Exemplarily, the final concentration may be, but is not limited to, any one of or a range between any two of 0.05 μmol/L, 0.06 μmol/L, 0.07 μmol/L, 0.08 μmol/L, 0.09 μmol/L, 0.1 μmol/L, 0.11 μmol/L, 0.12 μmol/L, 0.13 μmol/L, 0.14 μmol/L, and 0.15 μmol/L.

In some embodiments of the present disclosure, a final concentration of the sodium chloride in the sequencing primer reagent is 440 mmol/L-460 mmol/L. Exemplarily, the final concentration may be, but is not limited to, any one of or a range between any two of 440 mmol/L, 442 mmol/L, 444 mmol/L, 446 mmol/L, 448 mmol/L, 450 mmol/L, 452 mmol/L, 454 mmol/L, 456 mmol/L, 458 mmol/L, and 460 mmol/L.

In some embodiments of the present disclosure, a final concentration of the sodium citrate in the sequencing primer reagent is 40 mmol/L-50 mmol/L. Exemplarily, the final concentration may be, but is not limited to, any one of or a range between any two of 40 mmol/L, 41 mmol/L, 42 mmol/L, 43 mmol/L, 44 mmol/L, 45 mmol/L, 46 mmol/L, 47 mmol/L, 48 mmol/L, 49 mmol/L, and 50 mmol/L.

In some embodiments of the present disclosure, a volume fraction of the sodium dodecyl sulfate in the sequencing primer reagent is 0.01%-0.1%. Exemplarily, the volume fraction may be, but is not limited to, any one of or a range between any two of 0.01%, 0.02%, 0.03%, 0.04%, 0.05%, 0.06%, 0.07%, 0.08%, 0.09%, and 0.1%.

In some embodiments of the present disclosure, a volume fraction of the dextran sulfate in the sequencing primer reagent is 5%-15%. Exemplarily, the volume fraction may be, but is not limited to, any one of or a range between any two of 5%, 6%, 7%, 8%, 9%, 10%, 11%, 12%, 13%, 14%, and 15%.

In some embodiments of the present disclosure, a volume fraction of the formamide in the sequencing primer reagent is 25%-35%. Exemplarily, the volume fraction may be, but is not limited to, any one of or a range between any two of 25%, 26%, 27%, 28%, 29%, 30%, 31%, 32%, 33%, 34%, and 35%.

In some embodiments of the present disclosure, the hybridization treatment is performed at 36-40° C. for 15-25 min. Therefore, the specific hybridization of the sequencing adapter in the product of the ligation treatment to the sequencing primer can be ensured, thereby guaranteeing the accuracy of subsequent sequencing.

Exemplarily, the hybridization treatment is performed at 36° C., 37° C., 38° C., 39° C., or 40° C. for 15 min, 16 min, 17 min, 18 min, 19 min, 20 min, 21 min, 22 min, 23 min, 24 min, or 25 min.

In some embodiments, after the hybridization treatment is completed, the sequencing chip is rinsed sequentially with a first cleaning solution and a second cleaning solution to remove the residual sequencing primer reagent on the surface.

### Extension Treatment

After the hybridization treatment, as shown in step 6 in FIG. 1, the product of the ligation treatment serves as the template, and the hybridized sequencing primer serves as the primer. A reverse transcriptase or a mutant thereof, or other polymerases with both RNA- and DNA-dependent polymerase activities and mutants thereof, as well as dATP, are utilized to extend and complete the rU tail at the 3′ terminus of the 1^{st} cDNA in the product of the ligation treatment and the Poly(dT) on the surface of the sequencing chip that is not hybridized by RNA. The reason for extending and completing the Poly(dT) is to prevent the Poly(dT) ligated to the sequencing adapter from being sequenced in the process of subsequent sequencing, which would generate a background sequence. Therefore, the extension treatment is intended to fill the rU on the 1^{st} cDNA in one aspect, and to fill the redundant Poly(dT) in another aspect, and once filled, they are essentially excluded from being sequenced during sequencing.

Exemplarily, the reverse transcriptase may be M-MLV, and a polymerase with both RNA- and DNA-dependent polymerase activities may be Bst.

In this step, the extension treatment is performed in a filling reagent, and the filling reagent includes a reverse transcriptase, dATP, tris(hydroxymethyl)aminomethane hydrochloride, potassium chloride, magnesium chloride, dithiothreitol, and nuclease-free water.

In some embodiments of the present disclosure, a final concentration of the reverse transcriptase in the filling reagent is 5 U/μL-15 U/μL. Exemplarily, the final concentration may be, but is not limited to, any one of or a range between any two of 5 U/μL, 6 U/μL, 7 U/μL, 8 U/μL, 9 U/μL, 10 U/μL, 11 U/μL, 12 U/μL, 13 U/μL, 14 U/μL, and 15 U/μL.

In some embodiments of the present disclosure, a final concentration of the dATP in the filling reagent is 1 μmol/L-10 μmol/L. Exemplarily, the final concentration may be, but is not limited to, any one of or a range between any two of 1 μmol/L, 2 μmol/L, 3 μmol/L, 4 μmol/L, 5 μmol/L, 6 μmol/L, 7 μmol/L, 8 μmol/L, 9 μmol/L, and 10 μmol/L.

In some embodiments of the present disclosure, a final concentration of the tris(hydroxymethyl)aminomethane hydrochloride in the filling reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the potassium chloride in the filling reagent is 70 mmol/L-80 mmol/L. Exemplarily, the final concentration may be, but is not limited to, any one of or a range between any two of 71 mmol/L, 72 mmol/L, 73 mmol/L, 74 mmol/L, 75 mmol/L, 76 mmol/L, 77 mmol/L, 78 mmol/L, 79 mmol/L, and 80 mmol/L.

In some embodiments of the present disclosure, a final concentration of the magnesium chloride in the filling reagent is 1 mmol/L-5 mmol/L. Exemplarily, the final concentration may be, but is not limited to, any one of or a range between any two of 1 mmol/L, 2 mmol/L, 3 mmol/L, 4 mmol/L, and 5 mmol/L.

In some embodiments of the present disclosure, a final concentration of the dithiothreitol in the filling reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In order to completely extend and complete the 2-4 rUs at the 3′ terminus of the 1^{st} cDNA and the Poly(dT) on the surface of the sequencing chip that is not hybridized by RNA, in the process of the extension treatment, the filling reagent is introduced onto the surface of the sequencing chip in 2-5 batches. Exemplarily, the filling reagent may be introduced in 2 batches, 3 batches, 4 batches, or 5 batches.

In some embodiments of the present disclosure, the filling reagent is introduced in 2 batches.

The amount of the filling reagent introduced in batches is not specifically limited in the present disclosure, as long as the amount of the filling reagent introduced in the first batch can at least replace the reagent in the pipeline and the sequencing chip or fill the pipeline and the sequencing chip, and the amount of the filling reagent introduced in the second batch and the amount of the filling reagent introduced in the third batch can at least replace the filling reagent introduced into the sequencing chip in the previous batch. The more the filling reagent is introduced, the higher the replacement ratio and reaction efficiency will be. However, this also leads to increased reagent consumption and higher costs.

In some embodiments of the present disclosure, after each introduction of the filling reagent, the extension treatment is performed at 41-45° C. for 1-10 min. Therefore, the 2-4 rUs at the 3′ terminus of the 1^{st} cDNA and the Poly(dT) on the surface of the sequencing chip that is not hybridized by RNA can be completely extended and completed.

Exemplarily, the extension treatment is performed at 41° C., 42° C., 43° C., 44° C., or 45° C. for 1 min, 2 min, 3 min, 4 min, 5 min, 6 min, 7 min, 8 min, 9 min, or 10 min.

In some embodiments, after the extension treatment is completed, the sequencing chip is rinsed sequentially with a first cleaning solution and a second cleaning solution to remove the residual filling reagent on the surface.

### Sequencing Extension

After the extension treatment, as shown in step 7 in FIG. 1, the rU tail of the 3′ terminus of the 1^{st} cDNA in the product of the ligation treatment and the Poly(dT) on the surface of the sequencing chip that is not hybridized by RNA have been completed, and this prevents them from affecting the accuracy of the sequencing results.

In the process of the sequencing, a first cycle of sequencing extension is performed on the product of the extension treatment by using at least one of dTTP, dGTP, and dCTP as a substrate; then, subsequent sequencing extension is performed on the product of the first cycle of the sequencing extension by using at least one of dATP, dTTP, dGTP, and dCTP as a substrate. Since the T bases at the 3′ terminus of the 1^{st} cDNA have been completed with dATP (A pairs with T/U) in the process of the extension treatment, dATP may not be added in the first cycle of sequencing. In one embodiment, the substrate is dNTP modified with a fluorescent label and a reversible terminating group (N is selected from A, T, G, and C). Therefore, the type of the dNTP incorporated in each cycle of extension reaction is determined by detecting and analyzing the fluorescence signal of the dNTP incorporated in each cycle; after each cycle of extension reaction is completed, the reversible terminating group is cleaved off, allowing the extension reaction to proceed, ultimately achieving sequencing.

### Reagent Kit

The present disclosure provides a reagent kit. According to the embodiments of the present disclosure, the reagent kit includes a hybridization solution, a reverse transcription reagent, formamide, an rU tailing reagent, a PK reagent, a ligation reagent, a sequencing primer reagent, a filling reagent, a first cleaning solution, and a second cleaning solution. The reagent kit of the present disclosure makes the whole sequencing process more efficient. By directly capturing RNA molecules and performing reverse transcription and sequencing in a sequencing chip, no traditional library construction process is required, and the time-consuming PCR step of library construction is omitted, thereby saving a large amount of time and experimental resources. In addition, the reagent kit simplifies the manual operation steps in the process of library construction. By providing an optimized reagent combination, it enables experimental personnel to perform experiments more easily and reduces potential operational errors. This simplified operation design helps to improve the consistency and stability of the experiment. In addition, the integration of the reagent kit with the automation platform is good. This allows the whole sequencing process to be conveniently automated, improving the efficiency and reproducibility of the experiment. The design of the reagent kit takes the requirement for automated integration into consideration, and provides the experimental personnel with a convenient choice.

In some embodiments of the present disclosure, the sequencing method may further include at least one of the following additional technical features:

In some embodiments of the present disclosure, the reagent kit further includes a sequencing chip, and the surface of the sequencing chip is modified with Poly(dT) having a length of 40 nt-60 nt. Therefore, an RNA molecule with a Poly(A) tail at the 3′ terminus can be successfully captured.

Exemplarily, a length of the Poly(dT) may be any one of or a range between any two of 40 nt, 42 nt, 46 nt, 48 nt, 50 nt, 52 nt, 54 nt, 56 nt, 58 nt, or 60 nt.

In order to improve the hybridization stability of the RNA to the Poly(dT) probe on the surface of the sequencing chip, the Poly(dT) is provided with locked nucleic acid modifications. In some embodiments of the present disclosure, 1-5 locked nucleic acid modifications are introduced into the Poly(dT). The locked nucleic acid modifications can increase the Tm (DNA melting temperature) value of nucleic acid hybridization and promote the stability of the hybridized nucleic acid double-stranded structure; meanwhile, the modifications increase the hybridization temperature during probe capture and promote the formation of a longer and more stable hybridization region between the Poly(A) tail at the 3′ terminus of the RNA molecule and the Poly(dT) into which the locked nucleic acids are introduced. This results in more stable hybridization in the process of reverse transcription. Considering the sequence length of Poly(dT) as a capture probe, 1-5 locked nucleic acids are introduced into Poly(dT) for modification. Exemplarily, the number of locked nucleic acids introduced into Poly(dT) is 1, 2, 3, 4, or 5.

In some embodiments of the present disclosure, the reagent kit further includes at least one of a Poly(A) polymerase reaction buffer, nuclease-free water, a molecular crowding agent, an RNase inhibitor, ATP, T4-PNK, and a Poly(A) polymerase.

The Poly(A) polymerase reaction buffer, the nuclease-free water, the molecular crowding agent, the RNase inhibitor, the ATP, the T4-PNK, and the Poly(A) polymerase are used to add a Poly(A) tail to the 3′ terminus of an RNA molecule originally lacking a Poly(A) tail at its 3′ terminus (such as fragmented mRNA, miRNA, or cfRNA). The PK reagent is used to remove TdT adsorbed on the surface of the sequencing chip to prevent TdT from affecting the sequencing base signals. The first cleaning solution and the second cleaning solution are used to clean the sequencing chip after the completion of different steps.

The hybridization solution is used to capture an RNA molecule, so that the RNA molecule specifically hybridizes to Poly(dT) on the surface of the sequencing chip. In one example, the hybridization solution includes sodium chloride, sodium citrate, sodium dodecyl sulfate, dextran sulfate, and formamide. In this case, the capture of the RNA molecule by the Poly(dT) probe with locked nucleic acids can be promoted under the action of the hybridization solution, facilitating the hybridization of the RNA onto the surface of the sequencing chip.

In some embodiments of the present disclosure, a final concentration of the sodium chloride in the hybridization solution is 680 mmol/L-750 mmol/L. Exemplarily, the final concentration may be, but is not limited to, any one of or a range between any two of 680 mmol/L, 685 mmol/L, 690 mmol/L, 695 mmol/L, 700 mmol/L, 705 mmol/L, 710 mmol/L, 715 mmol/L, 720 mmol/L, 725 mmol/L, 730 mmol/L, 735 mmol/L, 740 mmol/L, 745 mmol/L, and 750 mmol/L.

In some embodiments of the present disclosure, a final concentration of the sodium citrate in the hybridization solution is 68 mmol/L-75 mmol/L. Exemplarily, the final concentration may be, but is not limited to, any one of or a range between any two of 68 mmol/L, 68.5 mmol/L, 69 mmol/L, 69.5 mmol/L, 70 mmol/L, 70.5 mmol/L, 71 mmol/L, 71.5 mmol/L, 72 mmol/L, 72.5 mmol/L, 73 mmol/L, 73.5 mmol/L, 74 mmol/L, 74.5 mmol/L, and 75 mmol/L.

In some embodiments of the present disclosure, a volume fraction of the sodium dodecyl sulfate in the hybridization solution is 0.05%-0.1%. Exemplarily, the volume fraction may be, but is not limited to, any one of or a range between any two of 0.05%, 0.06%, 0.07%, 0.08%, 0.09%, and 0.10%.

In some embodiments of the present disclosure, a volume fraction of the dextran sulfate in the hybridization solution is 14%-20%. Exemplarily, the volume fraction may be, but is not limited to, any one of or a range between any two of 14%, 15%, 16%, 17%, 18%, 19%, and 20%.

In some embodiments of the present disclosure, a volume fraction of the formamide in the hybridization solution is 45%-50%. Exemplarily, the volume fraction may be, but is not limited to, any one of or a range between any two of 45%, 46%, 47%, 48%, 49%, and 50%.

The reverse transcription reagent is used to obtain 1^{St} cDNA, that is, 1^{st} cDNA is obtained by taking the captured RNA molecule as a template and taking the Poly(dT) modified on the surface of the sequencing chip as a primer under the action of the reverse transcription reagent. The reverse transcription reagent includes a reverse transcriptase, dNTP, an RNase inhibitor, poloxamer 188, tris(hydroxymethyl)aminomethane hydrochloride, potassium chloride, magnesium chloride, dithiothreitol, and nuclease-free water.

In some embodiments of the present disclosure, a concentration of the reverse transcriptase in the reverse transcription reagent is 5 U/μL-15 U/μL. Exemplarily, the concentration may be, but is not limited to, any one of or a range between any two of 5 U/μL, 6 U/μL, 7 U/μL, 8 U/μL, 9 U/μL, 10 U/μL, 11 U/μL, 12 U/μL, 13 U/μL, 14 U/μL, and 15 U/μL.

In some embodiments of the present disclosure, a final concentration of the dNTP in the reverse transcription reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In some embodiments of the present disclosure, a final concentration of the RNase inhibitor in the reverse transcription reagent is 0.1 U/μL-1 U/μL. Exemplarily, the final concentration may be, but is not limited to, any one of or a range between any two of 0.1 U/μL, 0.2 U/μL, 0.3 U/μL, 0.4 U/μL, 0.5 U/μL, 0.6 U/μL, 0.7 U/μL, 0.8 U/μL, 0.9 U/μL, and 1 U/μL.

In some embodiments of the present disclosure, a volume fraction of the poloxamer 188 in the reverse transcription reagent is 5%-15%. Exemplarily, the volume fraction may be, but is not limited to, any one of or a range between any two of 5%, 6%, 7%, 8%, 9%, 10%, 11%, 12%, 13%, 14%, and 15%.

In some embodiments of the present disclosure, a final concentration of the tris(hydroxymethyl)aminomethane hydrochloride in the reverse transcription reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the potassium chloride in the reverse transcription reagent is 70 mmol/L-80 mmol/L. Exemplarily, the final concentration may be, but is not limited to, any one of or a range between any two of 71 mmol/L, 72 mmol/L, 73 mmol/L, 74 mmol/L, 75 mmol/L, 76 mmol/L, 77 mmol/L, 78 mmol/L, 79 mmol/L, and 80 mmol/L.

In some embodiments of the present disclosure, a final concentration of the magnesium chloride in the reverse transcription reagent is 1 mmol/L-5 mmol/L. Exemplarily, the final concentration may be, but is not limited to, any one of or a range between any two of 1 mmol/L, 2 mmol/L, 3 mmol/L, 4 mmol/L, and 5 mmol/L.

In some embodiments of the present disclosure, a final concentration of the dithiothreitol in the reverse transcription reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In order to improve the ligation efficiency of 1^{st} cDNA to a sequencing adapter, in the present disclosure, an rU tailing reagent is used to add 2-4 rU tails to the 3′ terminus of the 1^{st} cDNA obtained by reverse transcription. The rU tailing reagent includes TdT, UTP, tris(hydroxymethyl)aminomethane acetate, potassium acetate, magnesium acetate, CoCl₂, BSA, and nuclease-free water.

In some embodiments of the present disclosure, a final concentration of the TdT in the rU tailing reagent is 0.1 U/μL-0.3 U/μL. Exemplarily, the final concentration may be, but is not limited to, any one of or a range between any two of 0.1 U/μL, 0.2 U/μL, and 0.3 U/μL.

In some embodiments of the present disclosure, a final concentration of the UTP in the rU tailing reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In some embodiments of the present disclosure, a final concentration of the tris(hydroxymethyl)aminomethane acetate in the rU tailing reagent is 10 mmol/L-30 mmol/L. Exemplarily, the final concentration may be, but is not limited to, any one of or a range between any two of 10 mmol/L, 12 mmol/L, 14 mmol/L, 16 mmol/L, 18 mmol/L, 20 mmol/L, 22 mmol/L, 24 mmol/L, 26 mmol/L, 28 mmol/L, and 30 mmol/L.

In some embodiments of the present disclosure, a final concentration of the potassium acetate in the rU tailing reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the magnesium acetate in the rU tailing reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In some embodiments of the present disclosure, a final concentration of the CoCl₂ in the rU tailing reagent is 0.1 mmol/L-0.5 mmol/L. Exemplarily, the final concentration may be, but is not limited to, any one of or a range between any two of 0.1 mmol/L, 0.2 mmol/L, 0.3 mmol/L, 0.4 mmol/L, and 0.5 mmol/L.

In some embodiments of the present disclosure, a final concentration of the BSA in the rU tailing reagent is 0.05 mg/mL-0.15 mg/mL. Exemplarily, the final concentration may be, but is not limited to, any one of or a range between any two of 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, and 0.15 mg/mL.

A ligation reagent is used to ligate a sequencing adapter to the 3′ terminus of 1^{st} cDNA added with an rU tail, so as to facilitate the normal operation of subsequent sequencing. The ligation reagent includes T4 RNA ligase 1, a sequencing adapter, a molecular crowding agent, ATP, tris(hydroxymethyl)aminomethane hydrochloride, magnesium chloride, dithiothreitol, and nuclease-free water.

In some embodiments of the present disclosure, a final concentration of the T4 RNA ligase 1 in the ligation reagent is 0.1 U/μL-1 U/μL. Exemplarily, the final concentration may be, but is not limited to, any one of or a range between any two of 0.1 U/μL, 0.2 U/μL, 0.3 U/μL, 0.4 U/μL, 0.5 U/μL, 0.6 U/μL, 0.7 U/μL, 0.8 U/μL, 0.9 U/μL, and 1 U/μL.

In some embodiments of the present disclosure, a final concentration of the sequencing adapter in the ligation reagent is 0.5 μmol/L-1.5 μmol/L. Exemplarily, the final concentration may be, but is not limited to, any one of or a range between any two of 0.5 μmol/L, 0.6 μmol/L, 0.7 μmol/L, 0.8 μmol/L, 0.9 μmol/L, 1 μmol/L, 1.1 μmol/L, 1.2 μmol/L, 1.3 μmol/L, 1.4 μmol/L, and 1.5 μmol/L.

In some embodiments of the present disclosure, the sequencing adapter has the nucleotide sequence set forth in SEQ ID NO: 1.

(SEQ ID NO: 1)
5′p-TGGACATCTCGGGTGCCAAGGAACTCACGTCAC-ddC

In some embodiments of the present disclosure, a volume fraction of the molecular crowding agent in the ligation reagent is 15%-25%. Exemplarily, the volume fraction may be, but is not limited to, any one of or a range between any two of 15%, 16%, 17%, 18%, 19%, 20%, 21%, 22%, 23%, 24%, and 25%.

In some embodiments of the present disclosure, a final concentration of the ATP in the ligation reagent is 0.05 mmol/L-0.15 mmol/L. Exemplarily, the final concentration may be, but is not limited to, any one of or a range between any two of 0.05 mmol/L, 0.06 mmol/L, 0.07 mmol/L, 0.08 mmol/L, 0.09 mmol/L, 0.1 mmol/L, 0.11 mmol/L, 0.12 mmol/L, 0.13 mmol/L, 0.14 mmol/L, and 0.15 mmol/L.

In some embodiments of the present disclosure, a final concentration of the tris(hydroxymethyl)aminomethane hydrochloride in the ligation reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the magnesium chloride in the ligation reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In some embodiments of the present disclosure, a final concentration of the dithiothreitol in the ligation reagent is 0.5 mmol/L-1.5 mmol/L. Exemplarily, the final concentration may be, but is not limited to, any one of or a range between any two of 0.5 mmol/L, 0.6 mmol/L, 0.7 mmol/L, 0.8 mmol/L, 0.9 mmol/L, 1 mmol/L, 1.1 mmol/L, 1.2 mmol/L, 1.3 mmol/L, 1.4 mmol/L, and 1.5 mmol/L.

In order to hybridize an ssDNA sequence that is reversely complementary to the sequencing adapter sequence for use as a sequencing primer, the reaction is performed in a sequencing primer reagent. The sequencing primer reagent includes a sequencing primer, sodium chloride, sodium citrate, sodium dodecyl sulfate, dextran sulfate, formamide, and nuclease-free water.

In some embodiments of the present disclosure, the sequencing primer has the nucleotide sequence set forth in SEQ ID NO: 2.

(SEQ ID NO: 2)
GTGACGTGAGTTCCTTGGCACCCGAGATGTCCA

In some embodiments of the present disclosure, a final concentration of the sequencing primer in the sequencing primer reagent is 0.05 μmol/L-0.15 μmol/L. Exemplarily, the final concentration may be, but is not limited to, any one of or a range between any two of 0.05 μmol/L, 0.06 μmol/L, 0.07 μmol/L, 0.08 μmol/L, 0.09 μmol/L, 0.1 μmol/L, 0.11 μmol/L, 0.12 μmol/L, 0.13 μmol/L, 0.14 μmol/L, and 0.15 μmol/L.

In some embodiments of the present disclosure, a final concentration of the sodium chloride in the sequencing primer reagent is 400 mmol/L-500 mmol/L. Exemplarily, the final concentration may be, but is not limited to, any one of or a range between any two of 440 mmol/L, 442 mmol/L, 444 mmol/L, 446 mmol/L, 448 mmol/L, 450 mmol/L, 452 mmol/L, 454 mmol/L, 456 mmol/L, 458 mmol/L, and 460 mmol/L.

In some embodiments of the present disclosure, a final concentration of the sodium citrate in the sequencing primer reagent is 40 mmol/L-50 mmol/L. Exemplarily, the final concentration may be, but is not limited to, any one of or a range between any two of 40 mmol/L, 41 mmol/L, 42 mmol/L, 43 mmol/L, 44 mmol/L, 45 mmol/L, 46 mmol/L, 47 mmol/L, 48 mmol/L, 49 mmol/L, and 50 mmol/L.

In some embodiments of the present disclosure, a volume fraction of the sodium dodecyl sulfate in the sequencing primer reagent is 0.01%-0.1%. Exemplarily, the volume fraction may be, but is not limited to, any one of or a range between any two of 0.01%, 0.02%, 0.03%, 0.04%, 0.05%, 0.06%, 0.07%, 0.08%, 0.09%, and 0.1%.

In some embodiments of the present disclosure, a volume fraction of the dextran sulfate in the sequencing primer reagent is 5%-15%. Exemplarily, the volume fraction may be, but is not limited to, any one of or a range between any two of 5%, 6%, 7%, 8%, 9%, 10%, 11%, 12%, 13%, 14%, and 15%.

In some embodiments of the present disclosure, a volume fraction of the formamide in the sequencing primer reagent is 25%-35%. Exemplarily, the volume fraction may be, but is not limited to, any one of or a range between any two of 25%, 26%, 27%, 28%, 29%, 30%, 31%, 32%, 33%, 34%, and 35%.

In order to extend and complete 2-4 rUs added to the 3′ terminus of the 1^{st} cDNA in the product of the ligation treatment and the Poly(dT) on the surface of the sequencing chip that is not hybridized by RNA, a filling reagent is used for the extension treatment. The filling reagent includes a reverse transcriptase, dATP, tris(hydroxymethyl)aminomethane hydrochloride, potassium chloride, magnesium chloride, dithiothreitol, and nuclease-free water.

In some embodiments of the present disclosure, a final concentration of the reverse transcriptase in the filling reagent is 5 U/μL-15 U/μL. Exemplarily, the final concentration may be, but is not limited to, any one of or a range between any two of 5 U/μL, 6 U/μL, 7 U/μL, 8 U/μL, 9 U/μL, 10 U/μL, 11 U/μL, 12 U/μL, 13 U/μL, 14 U/μL, and 15 U/μL.

In some embodiments of the present disclosure, a final concentration of the dATP in the filling reagent is 1 μmol/L-10 μmol/L. Exemplarily, the final concentration may be, but is not limited to, any one of or a range between any two of 1 μmol/L, 2 μmol/L, 3 μmol/L, 4 μmol/L, 5 μmol/L, 6 μmol/L, 7 μmol/L, 8 μmol/L, 9 μmol/L, and 10 μmol/L.

In some embodiments of the present disclosure, a final concentration of the tris(hydroxymethyl)aminomethane hydrochloride in the filling reagent is 45 mmol/L-55 mmol/L. Exemplarily, the final concentration may be, but is not limited to, any one of or a range between any two of 45 mmol/L, 46 mmol/L, 47 mmol/L, 48 mmol/L, 49 mmol/L, 50 mmol/L, 51 mmol/L, 52 mmol/L, 53 mmol/L, 54 mmol/L, and 55 mmol/L.

In some embodiments of the present disclosure, a final concentration of the potassium chloride in the filling reagent is 70 mmol/L-80 mmol/L. Exemplarily, the final concentration may be, but is not limited to, any one of or a range between any two of 71 mmol/L, 72 mmol/L, 73 mmol/L, 74 mmol/L, 75 mmol/L, 76 mmol/L, 77 mmol/L, 78 mmol/L, 79 mmol/L, and 80 mmol/L.

In some embodiments of the present disclosure, a final concentration of the magnesium chloride in the filling reagent is 1 mmol/L-5 mmol/L. Exemplarily, the final concentration may be, but is not limited to, any one of or a range between any two of 1 mmol/L, 2 mmol/L, 3 mmol/L, 4 mmol/L, and 5 mmol/L.

In some embodiments of the present disclosure, a final concentration of the dithiothreitol in the filling reagent is 5 mmol/L-15 mmol/L. Exemplarily, the final concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In some embodiments of the present disclosure, the PK reagent includes proteinase K, tris(hydroxymethyl)aminomethane hydrochloride, sodium chloride, sodium dodecyl sulfonate, ethylenediaminetetraacetic acid disodium, and nuclease-free water.

In some embodiments of the present disclosure, a concentration of the proteinase K in the PK reagent is 0.05 mg/mL-0.15 mg/mL. Exemplarily, the concentration may be, but is not limited to, any one of or a range between any two of 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, and 0.15 mg/mL. Therefore, TdT on the surface of the sequencing chip can be completely removed, thereby ensuring the accuracy of subsequent sequencing results.

In some embodiments of the present disclosure, a final concentration of the tris(hydroxymethyl)aminomethane hydrochloride in the PK reagent is 95 mmol/L-105 mmol/L. Exemplarily, the final concentration may be, but is not limited to, any one of or a range between any two of 95 mmol/L, 96 mmol/L, 97 mmol/L, 98 mmol/L, 99 mmol/L, 100 mmol/L, 101 mmol/L, 102 mmol/L, 103 mmol/L, 104 mmol/L, and 105 mmol/L.

In some embodiments of the present disclosure, a final concentration of the sodium chloride in the PK reagent is 195 mmol/L-205 mmol/L. Exemplarily, the final concentration may be, but is not limited to, any one of or a range between any two of 195 mmol/L, 196 mmol/L, 197 mmol/L, 198 mmol/L, 199 mmol/L, 200 mmol/L, 201 mmol/L, 202 mmol/L, 203 mmol/L, 204 mmol/L, and 205 mmol/L.

In some embodiments of the present disclosure, a volume fraction of the sodium dodecyl sulfonate in the PK reagent is 0.05%-0.15%. Exemplarily, the volume fraction may be, but is not limited to, any one of or a range between any two of 0.05%, 0.06%, 0.07%, 0.08%, 0.09%, 0.1%, 0.11%, 0.12%, 0.13%, 0.14%, and 0.15%.

In some embodiments of the present disclosure, a concentration of the EDTA disodium in the PK reagent is 5 mmol/L-15 mmol/L. Exemplarily, the concentration may be, but is not limited to, any one of or a range between any two of 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, and 15 mmol/L.

In the sequencing method described above, a first cleaning solution is required for a first cleaning after the capture treatment, the reverse transcription treatment, the RNA template removal treatment, the uridylation treatment, the washing treatment of a PK reagent, the ligation treatment, the hybridization treatment, and the extension treatment, and the first cleaning solution includes sodium chloride, sodium citrate, 4-hydroxyethylpiperazine ethanesulfonic acid, and sodium dodecyl sulfate.

In some embodiments of the present disclosure, a final concentration of the sodium chloride in the first cleaning solution is 145 mmol/L-155 mmol/L. Exemplarily, the final concentration may be, but is not limited to, any one of or a range between any two of 145 mmol/L, 146 mmol/L, 147 mmol/L, 148 mmol/L, 149 mmol/L, 150 mmol/L, 151 mmol/L, 152 mmol/L, 153 mmol/L, 154 mmol/L, and 155 mmol/L.

In some embodiments of the present disclosure, a final concentration of the sodium citrate in the first cleaning solution is 10 mmol/L-20 mmol/L. Exemplarily, the final concentration may be, but is not limited to, any one of or a range between any two of 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, and 20 mmol/L.

In some embodiments of the present disclosure, a final concentration of the 4-hydroxyethylpiperazine ethanesulfonic acid in the first cleaning solution is 145 mmol/L-155 mmol/L. Exemplarily, the final concentration may be, but is not limited to, any one of or a range between any two of 145 mmol/L, 146 mmol/L, 147 mmol/L, 148 mmol/L, 149 mmol/L, 150 mmol/L, 151 mmol/L, 152 mmol/L, 153 mmol/L, 154 mmol/L, and 155 mmol/L.

In some embodiments of the present disclosure, a volume fraction of the sodium dodecyl sulfate in the first cleaning solution is 0.05%-0.15%. Exemplarily, the volume fraction may be, but is not limited to, any one of or a range between any two of 0.05%, 0.06%, 0.07%, 0.08%, 0.09%, 0.1%, 0.11%, 0.12%, 0.13%, 0.14%, and 0.15%.

In the sequencing method described above, a second cleaning solution is used for a second cleaning after the capture treatment, the removal of the RNA template, the washing treatment of a PK reagent, the hybridization treatment, the extension treatment, and the cleaning with the first cleaning solution, and the second cleaning solution includes sodium chloride and sodium citrate.

In some embodiments of the present disclosure, a final concentration of the sodium chloride in the second cleaning solution is 440 mmol/L-460 mmol/L. Exemplarily, the final concentration may be, but is not limited to, any one of or a range between any two of 440 mmol/L, 442 mmol/L, 444 mmol/L, 446 mmol/L, 448 mmol/L, 450 mmol/L, 452 mmol/L, 454 mmol/L, 456 mmol/L, 458 mmol/L, and 460 mmol/L.

In some embodiments of the present disclosure, a final concentration of the sodium citrate in the second cleaning solution is 40 mmol/L-50 mmol/L. Exemplarily, the final concentration may be, but is not limited to, any one of or a range between any two of 40 mmol/L, 41 mmol/L, 42 mmol/L, 43 mmol/L, 44 mmol/L, 45 mmol/L, 46 mmol/L, 47 mmol/L, 48 mmol/L, 49 mmol/L, and 50 mmol/L.

Examples of the present disclosure are described in detail below. The examples described below are exemplary and are merely used to explain the present disclosure, and they should not be construed as limiting the present disclosure. The examples without a specified particular technique or condition are performed in accordance with techniques or conditions described in the literature in the art or in accordance with the product instructions. Reagents or instruments without specified manufacturers used herein are conventional and commercially available products.

### Example 1: Hybrid Capture of RNA Using Flow Cell (Sequencing Chip)

Ribonucleic acid (RNA) may be total RNA, mRNA, miRNA, or cfRNA isolated and extracted from animals, plants, or microorganisms. Different types of RNA can all be sequenced using the method described in the present disclosure. However, when different types of RNA are obtained by extraction, their pretreatment methods generally differ. The specific procedures were as follows:

1. Fragmented mRNA Sample Processing and Hybrid Capture

(1) mRNA was enriched from 1 μg of RNA, eluted with 12.8 μL of nuclease-free water, and 10.8 μL of eluted product was collected for subsequent experiments.

(2) The 10.8 μL of eluted product was mixed with 1.2 μL of 10×Poly(A) polymerase reaction buffer, and the mixture was placed in a PCR instrument and fragmented under reaction at 94° C. for 5 min, followed immediately by incubation in an ice bath for more than 2 min to obtain a fragmented product.

(3) 12 μL of fragmented product was taken, and 1.8 μL of 10×Poly(A) polymerase reaction buffer, 0.9 μL of nuclease-free water, 12 μL of PEG8000 (50%), 0.375 μL of RNase inhibitor (40 U/μL), 0.225 μL of ATP (10 mmol/L), 1.2 μL of T4 PNK (10 U/μL), and 1.5 μL of Poly(A) polymerase (5 U/μL) were added, resulting in a total volume of 30 μL. After uniform mixing, the reaction was performed at 37° C. for 30 min and immediately terminated by adding 0.4 μL of 0.5 μmol/L EDTA disodium to obtain a Poly(A) product.

(4) 15 μL of Poly(A) product was mixed uniformly with 25 μL of hybridization solution (720 mM sodium chloride, 72 mM sodium citrate, 0.08% sodium dodecyl sulfate, 16% dextran sulfate, 48% formamide) to formulate 40 μL of hybridization sample mixture.

(5) Hybrid capture was performed in a Flow Cell using an X-bot sampler. First, the hybridization sample mixture was loaded into the Flow Cell (where the Poly(dT) in the Flow Cell contains 1-5 locked nucleic acids) using the X-bot sampler and incubated at 37° C. for 20 min. Subsequently, the unhybridized sample in the Flow Cell was cleaned using a first cleaning solution and a second cleaning solution. The components of the first cleaning solution were as follows: 150 mM sodium chloride, 15 mM sodium citrate, 150 mM 4-hydroxyethylpiperazine ethanesulfonic acid, and 0.1% sodium dodecyl sulfate (pH 7.0 at 25° C.); the components of the second cleaning solution were as follows: 450 mM sodium chloride and 45 mM sodium citrate (pH 7.0 at 25° C.).

2. miRNA Sample Processing and Hybrid Capture

(1) 1 pmol of miRNA was taken, and the volume was brought to 7.25 μL by adding NF—H₂O. The mixture was mixed uniformly with 1 μL of 10×Poly(A) polymerase reaction buffer, 1 μL of ATP (diluted to 0.2 mmol/L in advance), 0.25 μL of RNase inhibitor (40 U/μL), and 0.5 μL of Poly(A) polymerase (5 U/μL). The mixture was then reacted at 37° C. for 30 min. Immediately after the reaction was completed, 0.3 μL of 0.5 μmol/L EDTA disodium was added to terminate the reaction, and the mixture was placed on ice for later use. The initial amount of miRNA with a Poly(A) tail can be as low as 0.05 μmol, provided that the molar ratio of ATP to miRNA is maintained at 200:1.

(2) The Poly(A) product was diluted to 10 nM using NF—H₂O. Then, 1.6 μL of diluted library was mixed uniformly with 13.4 μL of NF—H₂O and 25 μL of hybridization solution to formulate 40 μL of hybridization sample mixture.

(3) Hybrid capture was performed in a Flow Cell using an X-bot sampler. First, the hybridization sample mixture was loaded into the Flow Cell using the X-bot sampler and incubated at 37° C. for 20 min. Subsequently, the unhybridized sample in the Flow Cell was cleaned using a first cleaning solution and a second cleaning solution. 3. Direct hybrid capture of mRNA from total RNA

(1) 300 ng of total RNA was added to 25 μL of hybridization solution (sodium chloride at a final concentration of 720 mM, sodium citrate at a final concentration of 72 mM, 0.08% sodium dodecyl sulfate, 16% dextran sulfate, and 48% formamide), and nuclease-free water (Thermo, 10977015) was added until the total volume reached 40 μL. The mixture was mixed uniformly to obtain a hybridization sample mixture.

(2) Hybrid capture was performed in a Flow Cell using an X-bot sampler. First, the hybridization sample mixture was loaded into the Flow Cell using the X-bot sampler and incubated at 37° C. for 20 min. Subsequently, the unhybridized sample in the Flow Cell was cleaned using a first cleaning solution and a second cleaning solution.

### Example 2: Surface 1^{st} cDNA Synthesis and Removal of RNA Template

Reverse transcription to synthesize 1^{st} cDNA and removal of the RNA template were performed in the Flow Cell using an X-bot autosampler. First, the temperature of the Flow Cell was raised to 42° C., and a reverse transcription reagent was introduced and incubated for 45 min to perform the reverse transcription reaction. After the reaction was completed, the Flow Cell was cleaned with a first cleaning solution. Subsequently, the temperature was raised to 55° C., formamide was introduced to remove the RNA template, and the Flow Cell was cleaned with a first cleaning solution and a second cleaning solution. The components of the reverse transcription reagent were as follows: a Maxima H Minus reverse transcriptase (Thermo, EP0751) at a final concentration of 10 U/μL, dNTP (Thermo, R1121) at a final concentration of 1 mM, a RiboLock RNase inhibitor (Thermo, E00381) at a final concentration of 0.5 U/μL, 10% poloxamer 188 (Aladdin, K434430), tris(hydroxymethyl)aminomethane hydrochloride at a final concentration of 50 mM, potassium chloride at a final concentration of 75 mM, magnesium chloride at a final concentration of 3 mM, and dithiothreitol at a final concentration of 10 mM, (pH 8.3 at 25° C.).

### Example 3: Addition of rU Tail to 3′ Terminus of 1^{st} cDNA and Removal of TdT

Addition of an rU tail to the 3′ terminus of the 1^{st} cDNA and removal of TdT were performed in the Flow Cell using an X-bot autosampler. First, an rU tailing reagent was introduced into the Flow Cell and reacted at 37° C. for 30 min. Subsequently, the Flow Cell was cleaned with a first cleaning solution. Next, a PK reagent was introduced and reacted at 55° C. for 5 min. Afterwards, the Flow Cell was cleaned sequentially with a first cleaning solution and a second cleaning solution. The components of the rU tailing reagent were as follows: TdT (NEB, M0315L) at a final concentration of 0.2 U/μL, UTP (Yeasen Biotechnology, 10131ES03) at a final concentration of 1 mM, tris(hydroxymethyl)aminomethane acetate at a final concentration of 20 mM, potassium acetate at a final concentration of 50 mM, magnesium acetate at a final concentration of 10 mM, CoCl₂ at a final concentration of 0.25 mM, and BSA at a final concentration of 0.1 mg/mL (pH 7.9 at 25° C.).

The components of the PK reagent were as follows: proteinase K (Sangon Biotech, B600452-0001) at a final concentration of 0.1 mg/mL, tris(hydroxymethyl)aminomethane hydrochloride at a final concentration of 100 mM, sodium chloride at a final concentration of 200 mM, 0.1% sodium dodecyl sulfonate, and EDTA disodium at a final concentration of 10 mM (pH 8.0 at 25° C.).

### Example 4: Ligation to Sequencing Adapter, Hybridization to Sequencing Primer, and Extension and Completion

Ligation to a sequencing adapter, hybridization to a sequencing primer, and extension and completion were performed in the Flow Cell using an X-bot autosampler. First, a ligation reagent was introduced and incubated at 25° C. for 60 min to perform the ligation reaction. Subsequently, the Flow Cell was cleaned with a first cleaning solution. Next, a sequencing primer reagent was introduced and incubated at 37° C. for 20 min to perform the hybridization reaction. Afterwards, the temperature of the Flow Cell was lowered to 25° C., and the Flow Cell was cleaned sequentially with a first cleaning solution and a second cleaning solution. After the cleaning was completed, a filling reagent was loaded and incubated at 42° C. for 10 min to perform the reaction of extension and completion. After the reaction was completed, the temperature of the Flow Cell was lowered to 25° C., and the Flow Cell was cleaned sequentially with a first cleaning solution and a second cleaning solution. The components of the ligation reagent were as follows: T4 RNA ligase 1 (NEB, M0204S) at a final concentration of 0.5 U/μL, 33mer_5′p_ddC (sequencing adapter, sequence: 5′p-TGGACATCTCGGGTGCCAAGGAACTCACGTCAC-ddC, synthesized by Sangon Biotech) at a final concentration of 1 μM, 20% PEG8000 (NEB, B1004SVIAL), ATP (NEB, P0756SVIAL) at a final concentration of 0.1 mM, tris(hydroxymethyl)aminomethane hydrochloride at a final concentration of 50 mM, magnesium chloride at a final concentration of 10 mM, and dithiothreitol at a final concentration of 1 mM (pH 7.5 at 25° C.).

The components of the sequencing primer reagent were as follows: 33mer_SP (sequencing primer, sequence: GTGACGTGAGTTCCTTGGCACCCGAGAT GTCCA, synthesized by Sangon Biotech) at a final concentration of 0.1 μM, sodium chloride at a final concentration of 450 mM, sodium citrate at a final concentration of 45 mM, 0.05% sodium dodecyl sulfate, 10% dextran sulfate, and 30% formamide (pH 7.0 at 25° C.).

The components of the filling reagent were as follows: a Maxima H Minus reverse transcriptase (Thermo, EP0751) at a final concentration of 10 U/μL, dATP (Thermo, R0141) at a final concentration of 5 μM, tris(hydroxymethyl)aminomethane hydrochloride at a final concentration of 50 mM, potassium chloride at a final concentration of 75 mM, magnesium chloride at a final concentration of 3 mM, and dithiothreitol at a final concentration of 10 mM (pH 8.3 at 25° C.).

### Example 5: Sequencing

Sequencing was performed using the sequencing reagent kit corresponding to the sequencing platform with reference to the description in the instructions.

At least one of dT*TP, dG*TP, and dC*TP, which are reversible terminating bases modified with a fluorophore, was used as a substrate to perform the first cycle of sequencing extension on the product of the extension and completion; at least one of dA*TP, dT*TP, dG*TP, and dC*TP, which are reversible terminating bases modified with a fluorophore, was used as a substrate to perform subsequent sequencing extension on the product of the first cycle of the sequencing extension. “dA*TP, dT*TP, dG*TP, and dC*TP” represent reversible terminating bases modified with a fluorophore, where * denotes the fluorophore.

### Example 6: Testing Experiment

To test the relative accuracy of this method for miRNA abundance detection, the inventors synthesized six miRNAs related to colorectal cancer (miR-223-5p, sequence: 5′p-CGUGUAUUUGACAAGCUGAGUU-3′OH (SEQ ID NO: 3); miR-223-3p, sequence: 5′p-UGUCAGUUUGUCAAAUACCCCA-3′OH (SEQ ID NO: 4); miR-92a-3p, sequence: 5′p-UAUUGCACUUGUCCCGGCCUGU-3′OH (SEQ ID NO: 5); miR-92a-2-5p, sequence: 5′p-GGGUGGGGAUUUGUUGCAUUAC-3′OH (SEQ ID NO: 6); miR-130a-5p, sequence: 5′p-GCUCUUUUC ACAUUGUGCUACU-3′OH (SEQ ID NO: 7); miR-130a-3p, sequence: 5′p-CAGUGCAAUGUUAAA AGGGCAU-3′OH (SEQ ID NO: 8)). They were mixed in a molar ratio of 1:10:100:1000:10000:100000, and different library construction initial amounts, namely 1 μmol, 0.5 μmol, 0.1 μmol, and 0.05 μmol, were set. Processing and sequencing were performed according to Examples 1 to 5, with four replicates for each library construction initial amount. For the obtained sequencing data, low-quality reads were first removed. Then, dustmasker (Sherstnev, Alexander et al. “Direct sequencing of Arabidopsis thaliana RNA reveals patterns of cleavage and polyadenylation.” Nature structural & molecular biology vol. 19,8 (2012): 845-52. doi:10.1038/nsmb.2345) was used to filter out low-complexity reads, followed by the use of cutadapt to remove terminal polyA tails. Reads with a length of 15-30 nt were retained. Finally, 18 M reads were sampled and aligned to the 6 miRNA reference sequences, and the alignment results were normalized using CPM (counts per million).

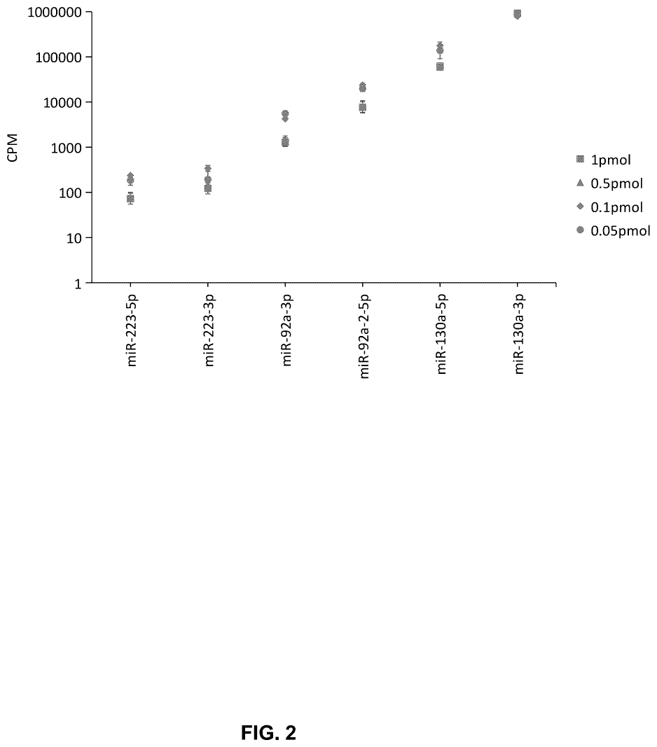

The results, as shown in FIG. 2, indicated a linear relationship across four orders of magnitude (from 100 to 1000000 CPM) when the library construction initial amount was 0.1 pmol to 1 μmol, demonstrating relatively accurate quantification. When the miRNA library construction initial amount was 0.1 μmol, the corresponding detection limit for miRNA was about 0.1 fmol (0.7 pg). When the number of target miRNAs was low or the miRNA library construction initial amount was less than 0.1 μmol, the quantification accuracy decreased.

### Example 7

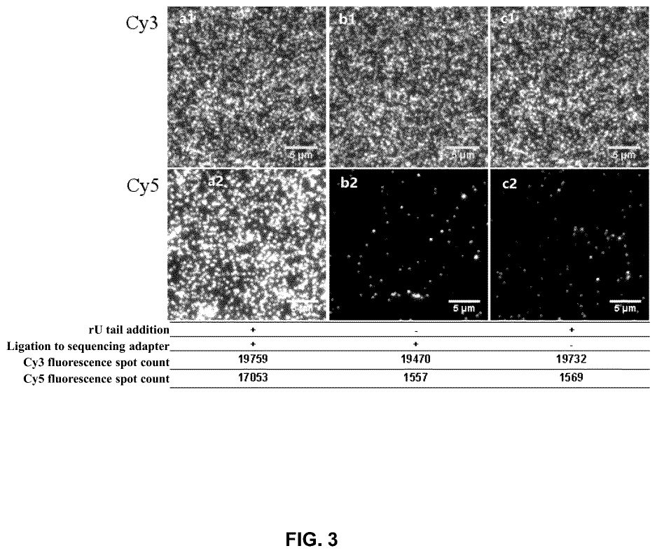

The ligation efficiency of the sequencing adapter on the surface of the Flow Cell was tested using a fluorescent probe hybridization method. A Cy3-modified receptor ssDNA was subjected to surface treatment with reference to the article: Zhao, Luyang et al. “Single molecule sequencing of the M13 virus genome without amplification.” PloS one vol. 12,12 e0188181. 18 Dec. 2017, doi:10.1371/journal.pone.0188181. Subsequently, the rU tail addition and sequencing adapter ligation reaction were performed with reference to Examples 3 and 4. After the reaction was completed, a Cy5-labeled fluorescent probe was hybridized to the sequencing adapter. Following hybridization, imaging was performed on a GenoCare1600 gene sequencer for observation. The Cy3 and Cy5 fluorescent spot counts were statistically analyzed, and the ligation efficiency of the ssDNA on the surface was estimated by calculating the ratio of Cy5 to Cy3 fluorescent spot counts. The results are shown in FIG. 3. The results showed that: the receptor ssDNA on the surface of the Flow Cell was successfully ligated to the sequencing adapter after rU tail addition and could be detected by hybridization to the Cy5-labeled fluorescent probe, with an overall efficiency of >85%. In contrast, the receptor ssDNA without rU tail addition or the receptor ssDNA with rU tail addition but without ligation to the sequencing adapter showed almost no hybridization to the Cy5-labeled fluorescent probe.

In the description of the specification, references to the terms such as “one embodiment”, “some embodiments”, “example” “specific example”, “some examples”, or the like, mean that specific features, structures, materials, or characteristics described in connection with the embodiment or example are included in at least one embodiment or example of the present disclosure. In the specification, the schematic expression of the terms described above does not necessarily refer to the same embodiment or example. Furthermore, the particular features, structures, materials, or characteristics may be combined in any suitable manner in any one or more embodiments or examples. In addition, in the absence of contradiction, those skilled in the art can combine the different embodiments or examples described in this specification or combine the features of different embodiments or examples.

Although embodiments of the present disclosure have been shown and described above, it would be appreciated by those of ordinary skill in the art that the embodiments described above are exemplary, cannot be construed to limit the present disclosure, and changes, modifications, substitutions, and variants can be made in the embodiments within the scope of the present disclosure.

What is claimed is:

1. A sequencing method, comprising:
performing capture treatment on an RNA molecule using a sequencing chip, wherein the surface of the sequencing chip is modified with Poly(dT), and the RNA molecule has a Poly(A) tail at the 3′ terminus;
performing reverse transcription treatment by using the captured RNA molecule as a template and Poly(dT) modified on the surface of the sequencing chip as a primer in the presence of dNTPs to synthesize 1^{st} cDNA, and then removing the captured RNA molecule;
performing uridylation treatment on the 3′ terminus of the 1^{st} cDNA to obtain uridylated 1^{st} cDNA having an rU tail at the 3′ terminus;
performing ligation treatment on the uridylated 1^{st} cDNA and the 5′ terminus of a sequencing adapter;
performing hybridization treatment on a product of the ligation treatment and a sequencing primer, wherein the sequencing primer is complementarily paired with at least a part of the sequencing adapter;
performing extension treatment on the sequencing primer by using the product of the ligation treatment as a template and the sequencing primer as a primer in the presence of dATP;
performing a first cycle of sequencing extension on a product of the extension treatment by using at least one of dTTP, dGTP, and dCTP as a substrate;
performing subsequent sequencing extension on a product of the first cycle of the sequencing extension by using at least one of dATP, dTTP, dGTP, and dCTP as a substrate.

2. The method according to claim 1, wherein the rU tail consists of 2-4 uridine nucleotides.

3. The method according to claim 2, wherein optionally, in the step of performing the uridylation treatment on the 3′ terminus of the 1^{st} cDNA, the uridylation is achieved by extending the 3′ terminus of the 1^{st} cDNA by 2-4 uracil uridine nucleotides by a terminal transferase.

4. The method according to claim 1, wherein the Poly(dT) has a locked nucleic acid modification.

5. The method according to claim 4, wherein, the Poly(dT) comprises 1-5 dTTPs having a locked nucleic acid modification.

6. The method according to claim 1, wherein the uridylation treatment is performed in an rU tailing reagent.

7. The method according to claim 6, wherein the rU tailing reagent comprises TdT, UTP, tris(hydroxymethyl)aminomethane acetate, potassium acetate, magnesium acetate, CoCl₂, BSA, and nuclease-free water.

8. The method according to claim 6, wherein a final concentration of the TdT in the rU tailing reagent is 0.1 U/μL-0.3 U/μL.

9. The method according to of claim 6, a final concentration of the UTP in the rU tailing reagent is 0.5 mmol/L-1.5 mmol/L.

10. The method according to claim 1, wherein the extension treatment is performed in a filling reagent.

11. The method according to claim 10, wherein the filling reagent comprises a reverse transcriptase, dATP, tris(hydroxymethyl)aminomethane hydrochloride, potassium chloride, magnesium chloride, dithiothreitol, and nuclease-free water.

12. The method according to claim 10, wherein, a final concentration of the reverse transcriptase in the filling reagent is 5 U/μL-15 U/μL;
optionally, a final concentration of the dATP in the filling reagent is 1 μmol/L-10 μmol/L.

13. A reagent kit, comprising: a hybridization solution, a reverse transcription reagent, formamide, an rU tailing reagent, a PK reagent, a ligation reagent, a sequencing primer reagent, a filling reagent, a first cleaning solution, and a second cleaning solution.

14. The reagent kit according to claim 13, further comprising a sequencing chip, wherein the surface of the sequencing chip is modified with Poly(dT).

15. The reagent kit according to claim 14, wherein, the Poly(dT) has a locked nucleic acid modification.

16. The reagent kit according to claim 15, wherein, the Poly(dT) has 1-5 locked nucleic acid modifications.

17. The reagent kit according to claim 13, wherein, the rU tailing reagent comprises TdT, UTP, tris(hydroxymethyl)aminomethane acetate, potassium acetate, magnesium acetate, CoCl₂, BSA, and nuclease-free water.

18. The reagent kit according to f claim 17, wherein, a final concentration of the TdT in the rU tailing reagent is 0.1 U/μL-0.3 U/μL.

19. The reagent kit according to f claim 17, wherein, a final concentration of the UTP in the rU tailing reagent is 0.5 mmol/L-1.5 mmol/L.

20. Use of a reagent kit in RNA sequencing, wherein the reagent kit, comprising: a hybridization solution, a reverse transcription reagent, formamide, an rU tailing reagent, a PK reagent, a ligation reagent, a sequencing primer reagent, a filling reagent, a first cleaning solution, and a second cleaning solution
